(19) United States
(12) Patent Application Publication
(10) Pub. No.: US 2025/0149145 A1
(43) Pub. Date: May 8, 2025

(54) PHYSICAL THERAPY ASSISTANT AS A SERVICE

(71) Applicant: Intel Corporation, Santa Clara, CA (US)

(72) Inventors: Sharon Talmor Marcovici, Kfar-Saba (IL); Rajasekaran Andiappan, Raseborg (FI); Dan Horovitz, Rishon Letzion (IL); Amit Gur, Ein Vered (IL); Lakshman Krishnamurthy, Portland, OR (US)

(73) Assignee: Intel Corporation, Santa Clara, CA (US)

(21) Appl. No.: 19/001,736

(22) Filed: Dec. 26, 2024

(51) Int. Cl. G16H 20/30

(52) U.S. Cl. CPC G16H 20/30

(57) ABSTRACT

Physical therapy assistant-as-a-service (PTaaS) enables the automatic evaluation of a patient's performance of physical therapy exercises and the automatic provision of feedback to the patient on their exercise performance in real-time. A patient device can provide real-time patient exercise video to a PTaaS backend that performs checks prior to the patient performing the exercise (pre-checks) and checks during patient performance of the exercise (live checks). If any of the checks fail, the PTaaS can provide feedback to the patient, such as if the patient is in an incorrect starting pose or has a body part at an incorrect angle before beginning the exercise or if the patient's form or posture during performance of the exercise needs to be adjusted. The PTaaS can automatically generate exercise metrics, reports, and physical therapy insights that a physical therapy clinician can access from a clinician portal.

### BACKGROUND

Physical therapy is a healthcare discipline focused on improving mobility, strength, and function through movement-based interventions. It uses techniques such as exercise and manual therapy to help individuals recover from injuries, manage chronic conditions, or prevent future physical impairments. Physical therapists assess each patient's unique needs and develop personalized treatment plans to address the patient's needs. Physical therapists typically oversee patient's performance of physical therapy exercises as they are learning them and during periodic visits to make sure they are performing the exercises correctly.

### BRIEF DESCRIPTION OF THE DRAWINGS

FIG. 1 illustrates an example architecture of a physical therapy assistant as a service (PTaaS) platform.

FIG. 2 is an example PTaaS kiosk.

FIG. 3 illustrates a first example patient application display output.

FIG. 4 illustrates a second example patient application display output.

FIG. 5 illustrates a third example patient application display output.

FIG. 6 illustrates a fourth example patient application display output.

FIG. 7 illustrates a fifth example patient application display output.

FIG. 8 illustrates an example clinician portal display output.

FIG. 9 is a first example method of a PTaaS platform providing real-time feedback to a patient performing physical therapy exercises.

FIG. 10 is a second example method of a PTaaS platform providing real-time feedback to a patient performing physical therapy exercises.

FIG. 11 is an example method of operating a clinician portal.

FIG. 12 is a block diagram of an example computing system in which technologies described herein may be implemented.

FIG. 13 is a block diagram of an example processor unit to execute computer-executable instructions as part of implementing technologies described herein.

### DETAILED DESCRIPTION

Physical therapy treatment typically involves a patient being the presence of a physical therapist. The patient can attend the physical therapist's clinic, or the physical therapist can meet the patient at their home or other location. By being in the patient's physical presence, the physical therapist can verify that the patient is performing prescribed exercises properly and assess their progress. In addition to the time spent participating in physical therapy sessions, a physical therapist's investment of resources in a patient includes the time spent preparing for the physical therapy sessions and documenting the patient's performance after the sessions. As physical therapy is an ongoing process that should continue when a patient leaves the clinic, an important part of physical therapy is the patient doing the home exercise program put together by their physical therapist. However, as important as performing these home exercises is, physical therapists do not have a way to track and analyze a patient's home exercise program performance. Further, when a patient performs a home exercise program on their own, they do not receive the feedback that they would receive from a physical therapist if they were performing the exercises in a clinical setting. Physical therapist feedback, which can comprise instructions on how to set up for and perform a new exercise, corrections on their form and posture while performing an exercise, and overall general encouragement is an important part of physical therapy treatment.

Described herein are technologies that enable a “physical therapy assistant as a service” (PTaaS) model for providing physical therapy services. The technologies disclosed herein provide an edge-to-edge closed loop from physical therapist exercise prescription to exercise performance by a patient away from the clinic to automatically providing feedback to the patient on their performance analysis of the patient's exercise performance to automatically providing exercise performance reports and physical therapy insights to the physical therapist. The PTaaS platform comprises a patient application, a camera to capture patient exercise activity, a clinician portal, and a backend that analyzes video of a patient's exercise performance (patient exercise video) and generates feedback to be provided to the patient in real-time. The patient application can operate on a patient's mobile computing device (e.g., smartphone, tablet, laptop computer) that also incorporates the camera used for recording patient exercise performance. The clinician portal allows a physical therapist to assign a treatment plan by assigning exercises to the patient for an in-clinic/home exercise program, view patient exercise performance videos, and view metrics and insights generated by the PTaaS backend.

The PTaaS backend performs pre-checks and live checks of patient exercise videos in real-time and provides real-time feedback to the patient application based on the results of the checks. Pre-checks are performed by the PTaaS backend before the patient starts an exercise (to ensure the patient is in a correct starting position, has a body part at a correct starting angle, is positioned properly with respect to the camera, etc.) and live checks are performed while the patient is performing an activity (to making sure the patient is performing the proper exercise, moving the correct limb, using proper form, holding proper posture, etc.). Various pre-checks and live checks can be associated with individual exercises and a clinician can select which checks are to be performed for an exercise when putting together an in-clinic/home exercise program. In some embodiments, the physical therapist can tailor an individual check for a patient by adjusting a goal for a check, such as a target knee angle for squats or a target shoulder angle for side lateral shoulder raises. While a patient is performing physical therapy exercises, patient exercise video is captured and streamed to the PTaaS backend in real-time. The backend performs the checks in real-time on the patient exercise video and determines whether feedback is to be provided to the patient.

The PTaaS backend can send real-time feedback on the patient exercise video to the patient application to be provided to the patient while they are performing their physical therapy exercises. The PTaaS backend operates in the cloud and thus can perform any number of checks on patient exercise video and provide feedback to the patient application in real-time. This immediate feedback can help ensure that patients are performing their prescribed physical therapy exercises correctly and safely. This kind of real-time feedback can be more effective than feedback received from a physical therapist who has reviewed patient exercise videos after an in-clinic/home exercise program session has been performed and sends feedback to the patient regarding their performance well after the fact. The PTaaS platform's ability to perform pre-checks and live checks in real-time creates a supportive environment that guides patients through their exercises with precision, enhancing the effectiveness of their treatment and ensuring their safety.

In addition to the advantage that providing real-time feedback to a patient performing physical therapy exercises in-clinic or at home can help them perform their exercises correctly and safely, the PTaaS technologies disclosed herein can have the following additional advantages. First, physical therapy with real-time feedback can now be done remotely, such as in a patient's home, workplace, or other non-clinical setting. This can free up both patient and clinician time by reducing the need for the patient to visit a physical therapy clinic to receive real-time feedback from a physical therapist, be assigned new exercises, and receive demonstrations on how the new exercises are to be performed. The patient benefits by saving the travel time to and from the clinic and the physical therapist benefits when the patient performs the exercises in-clinic, as the quality of the care received by the patient is not impacted while the physical therapist attends to other patients. The physical therapist further benefits from being able to offload patient exercise analysis and monitoring, and report generation to the PTaaS platform. The PTaaS service may thus be viewed as an artificial intelligence-based virtual physical therapy assistant. Third, the PTaaS platform stores patient data remotely and securely for offline analysis by physical therapists and artificial intelligence algorithms, and to provide patient exercise performance statistics, such as statistics that show the patient's exercise performance over time or the patient's exercise performance relative to other patients that have similar conditions. Fourth, by being a cloud-based service, the PTaaS backend can provide automated exercise feedback and analysis that has greater depth and breadth than what an edge computing device (e.g., patient device 105) can provide. By being cloud-based and not being limited by the computing resources of an edge computing device, a PTaaS platform can provide a richer physical therapy experience comprising detailed feedback relating to any number of checks associated with the exercises in a patient's in-clinic/home exercise program.

Fifth, a PTaaS platform may enable a more rigorous and thorough evaluation of patient exercise performance. A physical therapist may be focused on one aspect of a patient's exercise performance, such as increasing a patient's range of motion, and may fail to notice other deficiencies occurring simultaneously. Sixth, the PTaaS may be able to provide a level of quantitative feedback that a physical therapist may not be able to provide. The PTaaS platform can extract a body angle for every repetition in a set, for every set in an exercise, and for every exercise in a program. In a clinical setting, a physical therapist may use a goniometer to measure a body angle for only several repetitions of an exercise. The PTaaS platform can thus unlock a more analytical approach to assessing patient exercise performance and progress. Seventh, by being able to measure the body angles of a patient in real-time during exercise performance, the PTaaS can provide real-time feedback to a patient on whether they have reached an objective goal for each repetition of an exercise. For example, a patient no longer needs to wonder whether, for example, the depth of their squat is deep enough. If the goal for a squat exercise is for the knee angle to reach at least 90 degrees, the PTaaS platform can provide feedback (such as an audio beep or alert) when the repetition movement goal is met for each repetition. This can allow for patient exercise performance to be measured consistently against objective goals for every repetition in a program and across program sessions.

In the following description, specific details are set forth, but embodiments of the technologies described herein may be practiced without these specific details. Well-known circuits, structures, and techniques have not been shown in detail to avoid obscuring an understanding of this description. Phrases such as “an embodiment,” “various embodiments,” “some embodiments,” and the like may include features, structures, or characteristics, but not every embodiment necessarily includes the particular features, structures, or characteristics.

Some embodiments may have some, all, or none of the features described for other embodiments. “First,” “second,” “third,” and the like describe a common object and indicate different instances of like objects being referred to. Such adjectives do not imply objects so described must be in a given sequence, either temporally or spatially, in ranking, or in any other manner.

As used herein, the terms “operating”, “executing”, or “running” as they pertain to software or firmware in relation to a system, device, platform, or resource are used interchangeably and can refer to software or firmware stored in one or more computer-readable storage media accessible by the system, device, platform or resource, even though the software or firmware instructions are not actively being executed by the system, device, platform, or resource. The terms “comprising,” “including,” “having,” and the like, as used with respect to embodiments of the present disclosure, are synonymous.

Reference is now made to the drawings, which are not necessarily drawn to scale, wherein similar or same numbers may be used to designate same or similar parts in different figures. The use of similar or same numbers in different figures does not mean all figures including similar or same numbers constitute a single or same embodiment. Like numerals having different letter suffixes may represent different instances of similar components. The drawings illustrate generally, by way of example, but not by way of limitation, various embodiments discussed in the present document.

In the following description, for purposes of explanation, numerous specific details are set forth in order to provide a thorough understanding thereof. It may be evident, however, that the novel embodiments can be practiced without these specific details. In other instances, well-known structures and devices are shown in block diagram form in order to facilitate a description thereof. The intention is to cover all modifications, equivalents, and alternatives within the scope of the claims.

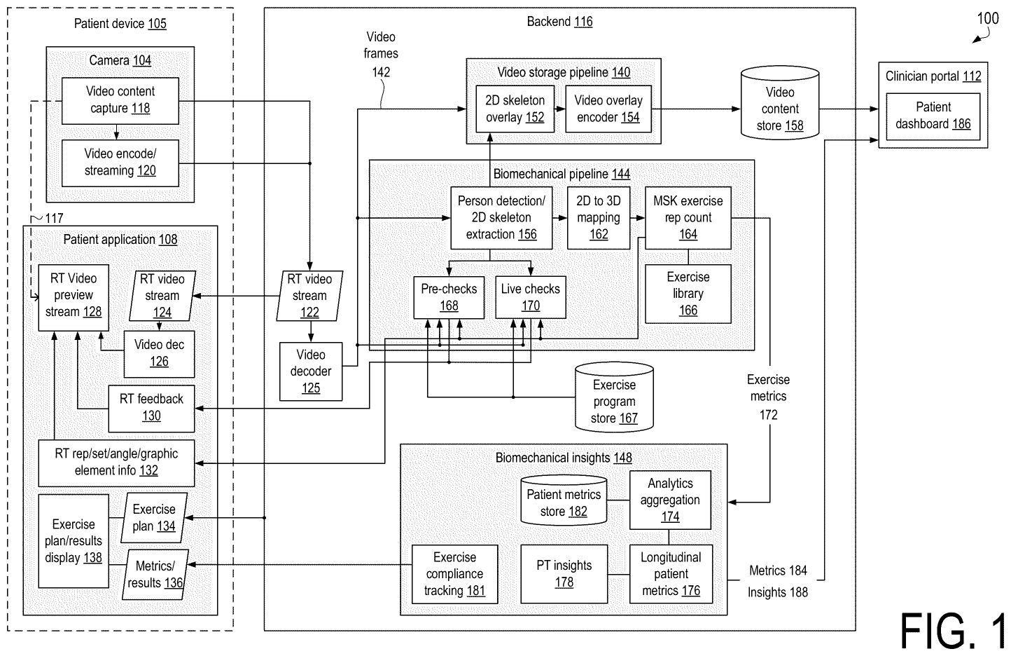

FIG. 1 illustrates an example architecture of a physical therapy assistant as a service (PTaaS) platform. The PTaaS platform 100 comprises a camera 104, a patient application 108, a clinician portal 112, and a PTaaS backend 116. A clinician (physical therapist) uses the clinician portal 112 to select physical therapy exercises for a patient to perform as part of an in-clinic/home exercise program. The camera 104 captures video of the patient getting ready to perform exercises and performing the exercises and streams the video (patient exercise video) in real-time to the PTaaS backend 116 for analysis. The PTaaS backend 116 performs checks on the patient exercise video in real-time, determines feedback to be provided to the patient, and sends the feedback to the patient application 108. The patient application 108 provides feedback to the patient in real-time so that the patient can correct their exercise performance on the fly. As discussed further below, the PTaaS backend 116 can also automatically generate exercise metrics, exercise reports, and physical therapy insights based on the patient exercise video. These reports and insights can be accessed by the clinician at the clinician portal 112.

The camera 104 comprises a video content capture module 118 that captures video of a patient performing a physical therapy exercise. The video content capture module 118 provides the patient exercise video to a video encoding and streaming module 120 that encodes the patient exercise video and streams the encoded video to the PTaaS backend 116. That is, the encoded video sent to the PTaaS backend 116 is representative of a patient's real-time ongoing performance of physical therapy exercises. The PTaaS backend 116 receives the real-time patient exercise video stream as real-time video stream 122. The PTaaS backend 116 forwards the real-time video stream 122 to the patient application 108 for display to the patient. The camera 104 can also stream real-time patient exercise video to the patient application 108, as discussed further below. The real-time patient exercise video can be streamed to the PTaaS backend 106 and/or the patient application 108 via the WebRTC or another suitable video streaming protocol.

The camera 104 can be integrated into or separate from a computing device on which the patient application 108 is operating. For example, in some embodiments, the camera 104 can be part of a patient device 105 that is executing on the patient application 108. The patient device 105, which could be a smartphone, tablet, laptop computer, or other suitable computing device. In such embodiments, the patient application 108 could receive patient exercise video from the camera 104 and not from the PTaaS backend 116. In some embodiments, the camera 104 can be a device that has received approval from the U.S. Food and Drug Administration (FDA) for use as a medical device. FDA-certified cameras meet the safety, accuracy, and reliability standards required for clinical applications. The patient device 105, clinician portal 112, administration portals, and camera 104 (if implemented as a separate device from the computing device executing patient application 108) can communicate with the PTaaS backend 116 via application program interfaces (APIs) over secure channels The device executing the patient application 108 (e.g., patient device 105) can be in real-time communication with the PTaaS backend 106 via WebSocket or another suitable data streaming protocol.

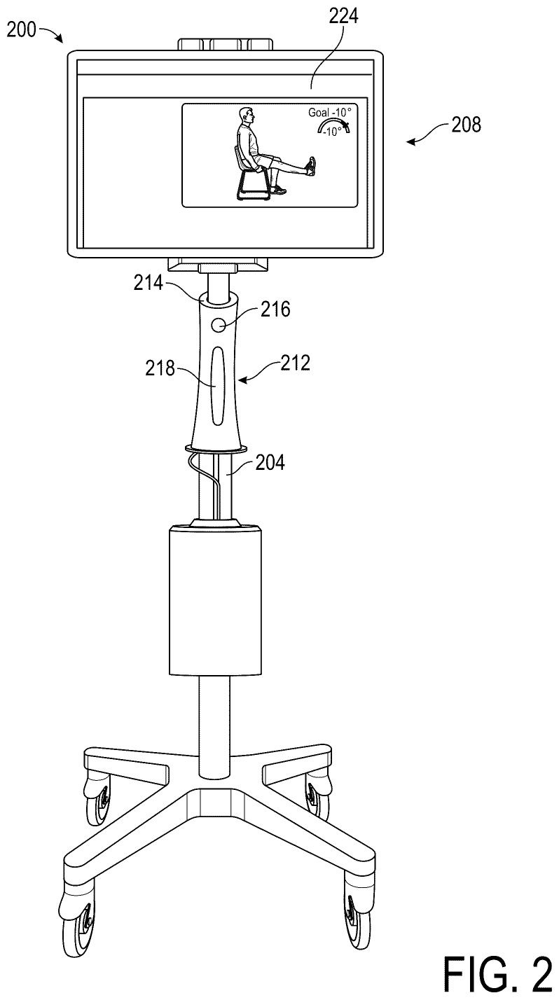

FIG. 2 is an example PTaaS kiosk. The kiosk 200 is designed for use in a clinical setting. The kiosk 200 comprises a stand 204 to which a computing device 208, shown as a tablet and a device 212 are removably attachable. The device 212 comprises a camera 216, a control button 218, visual indicators 214 (e.g., LEDs), and a speaker. The visual indicators 214 and speaker can be used to indicate the operational state and system errors of the device 212. The computing device 208 can run a patient application and display a preview of patient exercise video being streamed to a PTaaS backend on a display 224 of the computing device 208. In some embodiments, a kiosk management portal can manage the computing device 208 and the device 212. The kiosk management portal can perform such tasks as allowing certain patients and/or clinicians to use the computing device 208 and configuring the computing device 208.

Returning to FIG. 1, patient application 108 receives a real-time patient exercise video stream from the camera 104 (as indicated by line 117) or from the PTaaS backend 116 as real-time video stream 124. A video decoder 126 decodes the real-time video stream 124 to create a preview 128 of the real-time video. The display output displayed to a patient by patient application 108 (while they are performing physical therapy exercises) can include the preview 128 of the real-time video stream. The display output can further comprise information indicating the exercise being performed, the number of repetitions and sets completed, the number of sets to be completed, one or more body part angles, progress toward completion of a movement goal for one or more body parts for a repetition being performed, patient overlay graphic elements for one or more body parts, as well as additional information. The information can be overlaid on the real-time patient exercise video preview in the display output can be based on information continuously received in real-time by the patient application 108 from the PTaaS backend 116 while the patient is performing the exercise. This information can include including information indicating a number of completed repetitions for a current set, information indicating a number of completed sets for a current exercise, information indicating one or more body part angles, and information indicating graphic elements associated with one or more body parts to be overlaid on the patient or displayed in a vicinity of the patient (real-time repetition, set, body angle, and graphical clement information 132). The patient application 108 can provide real-time feedback to a patient in verbal, graphical, audio, and/or textual form. This real-time feedback can be based on information (feedback information 130) continuously received by the patient application 108 from the PTaaS backend 116. The real-time repetition, set, body angle, and graphical element information 132 and feedback information 130 are discussed in greater detail below. As used herein, the phrase “continuously received” means receiving information at a rate that allows for a patient application to generate display output comprising information based on the continuously received information such that a patient perceives the display output to track their exercise performance in real-time.

The exercise being performed by a patient and captured in the real-time video stream 122 can be part of an exercise plan stored at the PTaaS backend 116 and received by the patient application 108 as exercise plan information 134. The exercise plan information 134 can be provided to the patient application 108 by the PTaaS backend 116. The exercise plan information 134 can comprise information indicating updates to an exercise plan that have been made since a patient has last performed an exercise plan. The patient application 108 can further receive from the PTaaS backend 116 exercise metrics and results information 136 containing information about exercises that the patient has performed in the current and/or prior sessions, longitudinal metrics pertaining to the patient's exercise performance over time, or other exercise performance-based analytical results determined by the PTaaS backend 116. The patient application 108 can generate display output 138 that comprises any of the exercise plan information 134 and/or any of the metrics and results information 136.

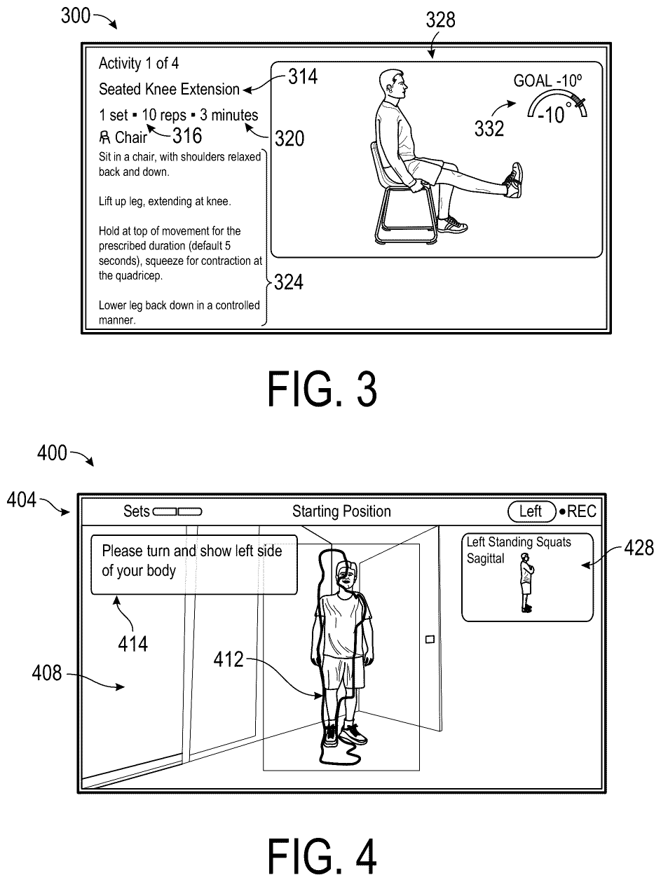

FIG. 3 illustrates a first example patient application display output. The display output 300 can be displayed to a patient before the start of an exercise that is part of an exercise program. The display output 300 comprises an exercise name 314, the number of sets and repetitions for each set 316 that are to be performed for the exercise, a goal rest time between sets 320, a description of the exercise 324, a video demonstration of the exercise 328, and a movement goal 332 for the exercise. In the display output 300, the movement goal is a knee angle of −10° (flexing the knee until the lower leg is within 10 degrees of parallel with the upper leg). In some embodiments, the display output 300 can display the length of time that a position (e.g., knee extended, arm raised) is to be held (e.g., 5, 10, or 30 seconds) and/or a time allotted to complete a set (e.g., 1, 2, or 3 minutes), in addition to or instead of a goal time between sets. In some embodiments, after completion of a set, the patient application can generate display output comprising a countdown timer counting down a target length of time the patient is to rest between sets.

The display output 300 can be shown on a display of any suitable device. As previously discussed, a patient application can execute on any suitable computing device, such as a smartphone, tablet, or laptop, and the display upon which any display output generated by a patient application is displayed can be integrated into the computing device executing the patient application, or a display external to and in communication with the computing device executing the patient application, such as a smart television or a wireless computer monitor. In some embodiments, the display on which the display output is displayed can be interactive (e.g., the display comprises a touchscreen).

FIG. 4 illustrates a second example patient application display output. The display output 400 comprises a status bar 404 and a preview 408 of real-time patient exercise video of a patient prior to performing an exercise. Various elements overlay the preview 408, including an inset 428 showing a starting position for the exercise. The display output 400 further comprises a body outline 412 that the patient is to align themselves with before starting the exercise and feedback 414 providing textual directions as to how the patient is to adjust their body before starting the exercise to align with the body outline 412. The body outline 412 and the feedback 414 can be based on information provided to the patient application 108 by the PTaaS backend 116 in response to the PTaaS backend 116 determining from patient exercise video that the patient's current position does not sufficiently match the starting position shown in inset 428.

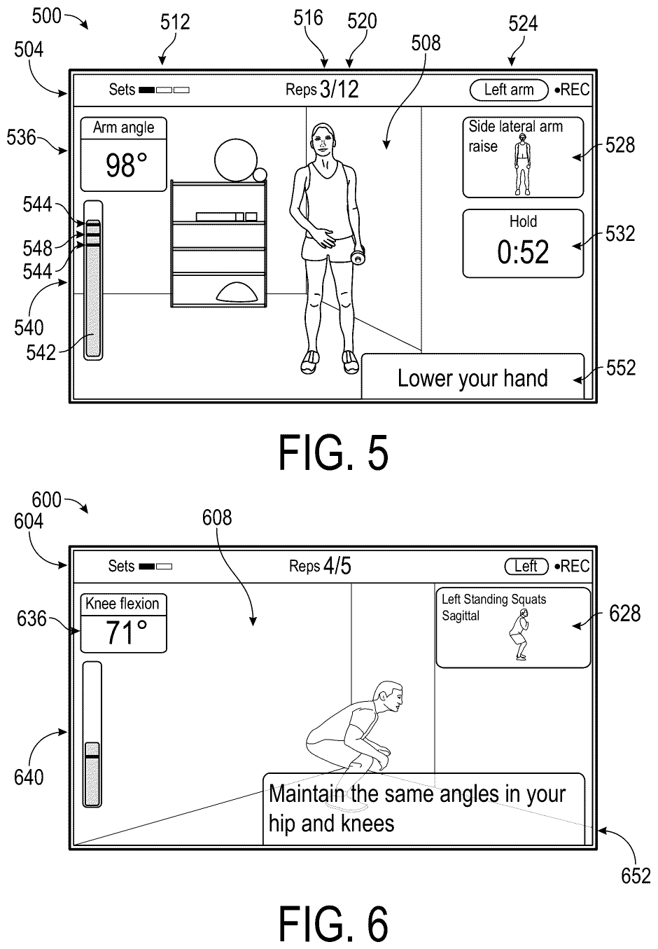

FIG. 5 illustrates a third example patient application display output. The display output 500 can be displayed during the performance of an exercise. The display output 500 comprises a status bar 504 and a preview 508 of real-time patient exercise video. The status bar 504 comprises an activity progress bar 512 showing how many sets of the current exercise are to be performed (the total number of graphic elements in the indicator) and how many sets have been completed (the number of shaded graphic elements in the indicator), the number of repetitions completed for the current set 516, the total number of repetitions to be performed in the current set 520, and text 524 indicating which part of the body is being exercised. In any of the display outputs described heroin, the number of completed repetitions for a current set displayed in a display output can be based on information indicating a number of completed repetitions for a current set continuously received by a patient application from a PTaaS backend. In any of the display outputs described herein, the number of sets completed as indicated in an activity progress bar can be based on information indicating a number of completed sets for a current exercise continuously received by a patient application from a PTaaS backend.

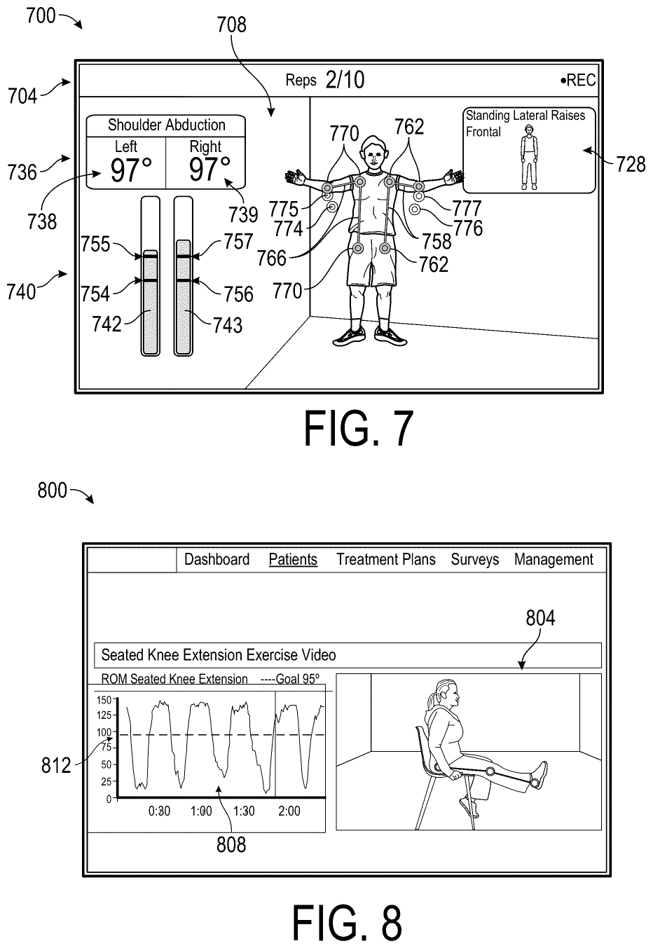

Various elements overlay the preview 508, including a demonstration video of the current exercise 528 (which may play after the patient begins performing the exercise) and a countdown timer 532 indicating how much longer the patient is to hold a position (e.g., arm raised, knee extended). The overlay elements further comprise an angle measurement indicator 536 that displays the current value of a body part angle (arm angle) relevant to the exercise being performed and a movement progress indicator 540 comprising a bar 542 that indicates an amount of movement of a body part. Outer marks 544 of the movement progress indicator 540 indicate a target body angle range (e.g., 80-100 degrees) with a center mark 548 indicating the middle of the target body angle range (e.g., 90 degrees). The angle displayed by the angle measurement indicator 536 and the movement progress displayed in movement progress indicator 540 are extracted by the PTaaS backend 116 from the real-time patient exercise video and sent to the patient application 108. For exercises involving the movement of more than one body part (e.g., both arms or both legs) the body part angle indicator and the movement progress indicator indicate the body angles and movement of multiple body parts (see FIG. 7).

In any of the display outputs described herein, the body part angle value displayed in an angle measurement indicator can be based on information indicating a body part angle continuously received by a patient application from a PTaaS backend. The patient application can determine the movement progress to be displayed in a movement progress indicator based on the information indicating a body part angle continuously received from the PTaaS backend.

The overlay elements displayed in FIG. 5 further comprise real-time textual feedback (textual feedback 552) to the patient based on their performance of the exercise. The textual feedback 552 can comprise feedback related to any of the checks (e.g., pre-checks, live checks) described herein, or any other exercise-related feedback (such as the next movement an exercise to perform (e.g., “now lower your arm”, “now extend your arm forward”) based on the patient's real-time performance of the exercise. The textual feedback 552 can provide general exercise feedback or feedback that is specific to an exercise. Here, the textual feedback 552 is exercise-specific feedback, instructing the patient to “lower your hand”.

FIG. 6 illustrates a fourth example patient application display output. The display output 600 can be displayed during the performance of an exercise. The display output 600 is similar to FIG. 5 in that it comprises a status bar 604, a preview 608 of real-time patient exercise video, a demonstration video 628 of the exercise being performed, angle measurement indicator 636, movement progress indicator 640, and textual feedback 652. The textual feedback 652, provided in real-time, provides exercise-specific feedback regarding the patient's form, encouraging them to “maintain the same angles in your hip and knees”.

FIG. 7 illustrates a fifth example patient application display output. The display output 700 can be displayed during performance of an exercise. The display output 700 comprises a status bar 704 and a preview 708 of the real-time video of a patient performing an exercise. Various elements overlay the preview 708, including a demonstration video of the current exercise 728, an angle measurement indicator 736, and a movement progress indicator 740. The angle measurement indicator 736 displays a right shoulder angle 738 and a left shoulder angle 739 and the movement progress indicator 740 comprises bars 742 and 743 indicating movement of the left shoulder and right shoulder, respectively, from their starting positions. Mark 754 indicates how much left shoulder movement is required for a left shoulder movement to count as a repetition and mark 755 indicates a goal for how much the left shoulder is to be moved. Similarly, mark 756 indicates how much right shoulder movement is required for a right shoulder movement to count as a repetition and a mark 757 indicates a goal for how much the right shoulder is to be moved.

The overlay elements further comprise graphical elements that overlay the patient or are displayed in the vicinity of the patient and correspond to the body part angles shown in the angle measurement indicator 736. In FIG. 7, lines 758 and nodes 762 correspond to the left shoulder angle 739 in the display output 700, and lines 766 and nodes 770 correspond to the right shoulder angle 738 displayed in the display output 700. The display output 700 further includes patient overlay graphic elements that are overlaid on the patient or displayed in the vicinity of the patient that indicate how much a patient is to move a body part within the patient's physical environment to have the movement count as a repetition or reach a movement goal. Node 776 indicates how much the patient is to raise their left arm to count as a repetition (corresponding to mark 754) and node 777 indicates how much the patient is to raise their left arm to reach a goal (corresponding to mark 755) set by the clinician. Node 774 indicates how much the patient is to raise their right arm to count as a repetition (corresponding to mark 756) and node 775 indicates how much the patient is to raise their right arm to reach a goal (corresponding to mark 757) set by the clinician.

In any of the display outputs described herein, the patient overlay graphic elements can be generated based on information continuously received by the patient application from the PTaaS backend indicating a graphic element associated with a body part of the patient to be overlaid on the patient or displayed in a vicinity of the patient.

The angles displayed in body part angle measurement indicators, the movement indicated in movement progress indicators, and position of the patient overlay graphic elements are based on measurements determined from information extracted from real-time patient exercise video by the PTaaS backend. Thus, body part angle information, movement progress information, and patient overlay graphic clement positions can change in real-time and track a patient's movements. The position of patient overlay graphic elements can change in real-time not only due to the patient moving to perform the exercise, but also as the user adjusts their overall body position (by way of moving to the left, right, front, or back) within the camera's field of view.

The display outputs illustrated in FIGS. 4-7 illustrate only several examples of real-time feedback that a PTaaS platform can provide to a patient as they set up for an exercise or as they are performing an exercise. The feedback can be in textual form, as illustrated in FIGS. 4-7 or in other forms. For example, in some embodiments, feedback can be provided in verbal form and can be provided in place of or in addition to textual feedback, with the verbal feedback providing the same or similar message as the textual feedback.

The feedback can be determined by the PTaaS backend in response to the PTaaS backend performing various checks on the real-time patient exercise video, which is discussed in greater detail below. As mentioned above, these checks comprise pre-checks and live checks. Generally, the feedback provided in response to a PTaaS backend performing a pre-check or live check is corrective (to get the patient in the correct position before starting an exercise, to point out deficiencies in their execution of an exercise, etc.).

Pre-checks are verifications performed before the start of an exercise. These pre-checks are performed to ensure that the patient is in a proper environment and position before beginning an exercise. The pre-checks that can be performed by the PTaaS backend include light range, patient position, camera distance, plane alignment, side orientation, occlusion, starting pose, and starting angle checks.

The light range pre-check assesses lighting conditions in the real-time patient exercise video to ensure that there is not too little or too much light. In some embodiments, the light range pre-check can determine whether the lux in the video is lower than a lower lighting level threshold (e.g., about three hundred lux) or greater than an upper lighting level threshold (e.g., 10,000, 20,000, or 30,000 lux, or another value representing the camera being in direct sunlight or otherwise receiving too much sunlight for the PTaaS backend to analyze the real-time video). As a result of performing the light range pre-check, if the PTaaS backend determines that the lighting brightness in the real-time patient exercise video is too low or too high, the PTaaS backend can provide the patient application with information indicating feedback to be provided to the patient indicating poor lighting conditions. The feedback can comprise an appropriate message, such as “too dark” or “too bright”.

The patient position pre-check verifies that the patient is positioned correctly within the camera field of view. As a result of performing the patient position pre-check, if the PTaaS backend determines that the patient is not positioned property with the camera field of view, the PTaaS backend can provide the patient application with feedback information indicating feedback to be provided to the patient indicating the patient is to move left or right. The feedback can comprise an appropriate message, such as “move left” or “move right”.

The camera distance pre-check verifies that the patient is positioned at a proper distance from the camera. As a result of performing the camera distance pre-check, if the PTaaS backend determines that the patient is not located at a proper distance from the camera, the PTaaS backend can provide to the patient application information indicating feedback to be provided to the patient indicating the patient is to move forward or backward. The feedback can comprise an appropriate message, such as “move forward” or “take a step back”.

The plane alignment pre-check verifies that the patient's proper anatomical plane (e.g., sagittal, coronal) for the exercise about to be performed is oriented parallel to the camera. As a result of performing the plane alignment pre-check, if the PTaaS backend determines that the correct anatomical plane of the patient is not aligned with the camera, the PTaaS backend can provide the patient application with information indicating feedback to be provided to the patient indicating the patient is to orient themselves so that the proper anatomical plane is oriented to the camera. The feedback can comprise an appropriate message, such as “turn and show the right side of your body”, “turn and show the left side of your body”, or “turn to show the front side of your body.”

The side orientation pre-check verifies that the patient's correct side for the exercise about to be performed is showing to the camera. As a result of performing the side orientation pre-check, if the PTaaS backend determines the patient is not showing the correct body side to the camera, the PTaaS backend can provide the patient application with information indicating feedback to be provided to the patient indicating the patient is to orient themselves so that the correct side of their body is facing the camera. The feedback can comprise an appropriate message, such as “incorrect side”, “turn to show your left side” or “turn to show your right side”.

The occlusion pre-check determines whether the patient is occluded by an object or another person. As a result of performing the occlusion pre-check, if the PTaaS backend determines that the patient is at least partially occluded, the PTaaS backend can provide the patient application with information indicating feedback to be provided to the patient indicating the patient is at least partially occluded. The feedback can comprise an appropriate message, such as “occlusion detected”, “remove obstructions”, or “please move so that your entire body is viewable by the camera”.

The starting pose pre-check determines whether the patient is in the correct starting pose (e.g., standing, sitting, supine, prone, side-lying, quadruped) for the exercise about to be performed. As a result of performing the starting pose pre-check, if the PTaaS backend determines that the patient is in an incorrect starting pose, the PTaaS backend can provide the patient application with information indicating feedback to be provided to the patient indicating the patient is to position themselves in the starting pose for the exercise. The feedback can comprise an appropriate message, such as “assume a sitting position”, “lie on your back”, “lie on your stomach”, “assume a standing position”, “get on your hands and needs”, “lie or your side”, or “spread your feet apart to shoulder width”.

The starting angle pre-check verifies that an angle of a patient's body part is at a proper starting angle for the exercise about to be performed. As a result of performing the starting angle pre-check, if the PTaaS backend determines that a body part of the patient is not at a starting angle, the PTaaS backend can provide the patient application with information indicating feedback to be provided to the user indicating the user is to position the body part at the starting angle. The feedback can comprise an appropriate message, such as “stand up straight”, “stand tall”, “hold your arms down at your side”, or “fully extend your knee”.

The PTaaS backend can perform additional checks or tasks related to the start of an exercise, such as confirming the start of an activity, performing error handling, and determining whether the patient's clothes conform to guidelines that ensure the PTaaS backend can perform exercise tracking. Once the PTaaS backend determines that a real-time patient exercise video passes all pre-checks, it can send information to the patient application indicating a message informing the patient that they can begin the exercise, such as “good to start” or “begin exercise”. If the PTaaS backend determines that any pre-checks of real-time patient exercise video have failed, the PTaaS backend may not analyze real-time patient exercise video until the errors are resolved. The PTaaS backend can perform a clothing guideline check as part of its pre-checks to ensure that a patient's clothing satisfies clothing guidelines to allow the PTaaS backend to track and analyze a patient's exercise performance. The PTaaS backend can provide information to the patient application indicating a message to be provided to the patient indicating that the clothes they are wearing do not allow for proper exercise tracking, such as “pants too loose for exercise tracking”, or “cannot detect body parts for exercise tracking, consider changing clothes”.

Once a patient has started performing an exercise, the PTaaS backend can perform live checks on the real-time patient exercise video. Live checks that can be performed by the PTaaS backend are checks performed in real-time while a patient performs an activity to ensure that the activity is being performed correctly and safely. This can maximize the efficacy of the exercises and reduce the risk of patient injury. The live checks that can be performed by the PTaaS platform include correct limb movement, correct exercise, and form and posture checks. In some embodiments, the live checks module can also perform gait analysis.

The correct limb live check determines whether the patient is moving the correct arm or leg for the exercise. As a result of performing the correct limb live check, if the PTaaS backend determines that the patient is moving the wrong limb, the PTaaS backend can provide the patient application with information indicating feedback to the user indicating the wrong limb is being moved. The feedback can comprise an appropriate message, such as “you should be moving your left arm in this exercise”, “move your other arm for this exercise”, or “move your right leg instead”.

The correct exercise live check determines whether the patient is performing an exercise out of sequence in an exercise program or an exercise that is not part of the exercise program. As a result of performing the correct exercise chase live check, if the PTaaS backend determines that the patient is not performing the correct exercise, the PTaaS backend can provide the patient application with information indicating feedback to the user indicating that they are not performing the correct exercise. The feedback can comprise an appropriate message, such as “wrong exercise”, “you still have one more set of squats to do” or “you should be performing shoulder lateral raises”.

The form and posture live check analyzes the real-time patient exercise video to ensure that the patient is using proper form and maintaining good posture while an exercise is being performed. As a result of performing the form and posture live check, if the PTaaS backend determines that the patient is using improper form, has incorrect posture for the exercise, or is performing the exercise is an incorrect pace, the PTaaS backend can provide the patient application with information comprising descriptive (or qualitative) feedback to the user indicating an adjustment to be made to the pace at which the user is performing the exercise or the user's form or posture of the user, such as “please slow down the repetition rate”, “please increase the repetition rate”, “keep hips perpendicular to floor”, “keep your arm straight”, “lower your hand”, “maintain the same angles in your hip and knees”, “move your knees straight up and down”, “raise your hips”, “try not to lean forward”, “keep your body straight”, “try not to allow your knees from collapsing inward”, “keep your knee aligned over your ankle”, “please straighten both legs”, “straighten your exercising leg”, “keep your other leg straight”, “keep your other leg straight”, “keep other leg aligned with upper body”, “keep hips perpendicular to upper body”, “move both arms together”, “keep legs folded as shown in the video”, “keep arms folded as shown in the video”, “make sure both legs are aligned”, “keep your other leg bent”, “don't lift your head”, “keep your back flat on the surface”, “keep your pelvis as shown in the video”, “don't go over shoulder height”, “keep your arm aligned with your body”, “keep elbow bent at 90 degrees”, or “don't roll your lower back”.

In some embodiments, the feedback provided to the patient to correct a form or posture deficiency can comprise quantitative information extracted from the real-time patient exercise video by the PTaaS backend, such as a quantitative adjustment to the movement of a body part to correct a form or posture deficiency. For example, a patient application could provide feedback messages such as “bring your knees together by two inches”, “your knees are moving out by 10 degrees when you are squatting—keep them together”, or “you are leaning forward by 20 degrees—try to stand straight”.

In some embodiments, the feedback provided by the PTaaS platform to attempt to correct a form or posture deficiency can comprise prescriptive feedback-suggestions or instructions on how the client can modify their performance of the exercise. For example, to address the exercise performance deficiencies of a patient rolling their lower back or lifting their heels, instead of providing descriptive feedback such as “don't roll your lower back” or “try not to lift your heels”, prescriptive feedback such as “engage your core” or “think of pushing through your heels” could be provided. The PTaaS backend could provide such prescriptive feedback after initially providing descriptive feedback and subsequently detecting in the real-time patient exercise video that the patient's continued performance of the exercise exhibits the same deficiency.

The form and posture feedback examples provided above are not an exhaustive list of the feedback that can be provided to correct form or posture deficiencies-they are merely a representative list. Feedback pertaining to the form and posture live check can comprise variations of the messages listed above and any other feedback that can aid a patient in correcting a form or posture deficiency during the performance of an exercise. Further, form and posture feedback need not be corrective. The PTaaS backend can comprise feedback information to the patient application indicating encouragement to be provided to the patient such as, “movement goal reached for every repetition in this set—great job!” or “all repetitions all performed successfully!”

In some embodiments, the real-time feedback provided by a PTaaS platform can be provided to a patient as soon as the PTaaS backend detects the form or posture deficiency. In some embodiments, real-time feedback can be provided after the completion of a repetition or after the completion of a set. In some examples, real-time feedback provided by a PTaaS platform can comprise audio feedback or cues, such as a beep or other sound to indicate the completion of a repetition, set, position hold time, rest time between sets, etc.

The PTaaS backend can perform additional checks related to the start of an exercise or during the performance of an exercise, such as multiple person and patient presence checks. As a result of performing a multiple person check, if the PTaaS backend determines from the real-time patient exercise video that multiple people are in the camera's field of view, the PTaaS backend can provide the patient application with information indicating feedback to be provided to the user indicating multiple people are detected in the camera's field of view. The feedback can comprise an appropriate message, such as “multiple people detected” or “please ensure that only one person is in the camera field of view”. As a result of performing a patient presence check, if the PTaaS backend determines from the real-time patient exercise video that the patient is no longer in the camera's field of view, the PTaaS backend can provide to the patient application information indicating feedback to be provided to the user indicating the patient is not in the camera's field of view. The feedback can comprise an appropriate message, such as “user no longer detected” or “please make sure that you are viewable by the camera”.

Further, if the patient application experiences or detects an error during performance of the exercise, the patient application can instruct the patient to stop the exercise and offer the patient options to start over or skip the exercise. The patient application can report the error to the PTaaS backend. In some embodiments, the PTaaS backend can send information to the patient application to stop the exercise if the PTaaS backend detects or experiences an error while analyzing real-time patient exercise video.

Returning to FIG. 1, the PTaaS backend 116 can be hosted by one or more computing devices located remotely from a patient device 105 or other computing devices that can execute a patient application 108. The patient device 105 or other computing devices that can execute a patient application 108 can connect to the PTaaS backend 116 via the Internet. The PTaaS backend host devices could be located in, for example, an on-premises computing facility or be part of a cloud service provider infrastructure. In some embodiments, the PTaaS backend 106 is a microservices architecture distributed via a software-as-a-service (SaaS) model, which enables scalability according to PTaaS system usage demand. A SaaS model provides robustness to any underlying system resource failure or overutilization due to its high availability (HA) and redundancy nature. The SaaS model of delivery thus ensures that the provision of physical therapy services via the PTaaS platform will not be impacted due to resource failures or deficiencies. The PTaaS backend can further leverage HIPAA (Health Insurance Portability and Accountability Act)-compliant cloud service provider services to guarantee health data consistency and privacy. Access to a PTaaS platform can be controlled by authorization and authentication mechanisms provided by a health care or cloud service provider. PTaaS platforms can comply with high-security standards (e.g., SOC2 (System and Organization Control 2)) and provide secure access-controlled authentication and authorization mechanisms, to provide least-privileges access to health data (including streamed and recorded videos, real-time and post-exercise metrics, and patient personal and health data).

The PTaaS backend 116 comprises a video decoder 125, a video storage pipeline 140, a biomechanical pipeline 144, and a biomechanical insights module 148. The video decoder 125 decodes the real-time video stream 122 received from a camera 104 or patient device 105 and provides the decoded real-time patient exercise video, in the form of video frames 142 (e.g., video frames encoded in RGB (red-green-blue format) or H.264 format), to the video storage pipeline 140 and the biomechanical pipeline 144.

The video storage pipeline 140 comprises a 2D skeleton overlay module 152 and a video overlay encoder 154. The 2D skeleton overlay module 152 can add a 2D skeleton overlay in the video frames. A 2D skeletal overlay is a graphical representation of the body (or at least a portion thereof) of the patient as a set of connected nodes or “joints” that correspond to anatomical features, such as the head, shoulders, elbows, wrists, hips, knees, and ankles. This “skeleton” overlays the patient's body in the video and mimics the body's movement. The 2D skeleton overlay module 152 can receive information indicating the location of a face in the video frames and information indicating the 2D skeleton that is to be added in the video frames, respectively, from a person detection/2D skeleton extraction module 156 in the biomechanical pipeline 144. The video overlay encoder 154 encodes the patient exercise video overlaid with a 2D skeleton. The encoded videos are stored in a secure video content store 158 with access restriction. The video content store 158 can have a high degree of resiliency owing to the PTaaS backend 106 being hosted in a cloud environment. In some embodiments, the encoded videos are encrypted. Clinicians and patients can gain access to the video content store 158 from clinician portals 112 and patient applications 108, respectively.

The biomechanical pipeline 144 comprises the person detection/2D skeleton extraction module 156, a 2D-to-3D mapping module 162, a musculoskeletal exercise repetition count module (MSK exercise repetition count module 164), an exercise library 166, a pre-checks module 168, and a live checks module 170. The person detection/2D skeleton extraction module 156 can also determine the location of a patient's body parts and generate information indicating a 2D skeleton overlay to be added to the video frames 142. This 2D skeleton information is provided to the 2D skeleton overlay module 152. The 2D-to-3D mapping module 162 maps the 2D skeleton extracted from the video frames 142 by the person detection/2D skeleton extraction module 156 to three-dimensional (3D) space. This can allow for the analysis of exercises where the patient is moving body parts in a motion that extends beyond a plane parallel to the camera, such as a patient facing towards the camera and performing shoulder horizontal abduction and adduction movements (i.e., extending an arm out to the side, perpendicular to the body, and then moving the arm horizontally across the body toward the midline of the body (adduction) and then back to the side (abduction)). The 2D-to-3D mapping module 162 can also enable the analysis of complex motions that have both a 2D and 3D component. In some embodiments, the 2D-to-3D mapping module 162 can employ deep learning models to perform the 2D-to-3D mapping. The 2D-to-3D mapping module 162 can communicate with the camera for camera calibration purposes.

The MSK exercise repetition count module 164 can determine body part angles, the amount a body part has moved from a starting position, the amount of a body part has moved relative to a movement goal, whether the movement of a body part counts as completion of a repetition, and/or information indicating a graphic element associated with a body part of the patient to be overlaid on the patient or displayed in a vicinity of the patient to the patient application 108, the pre-checks module 168, the live checks module 170, and the biomechanical insights module 148 (as exercise metrics 172). The MSK exercise repetition count module 164 can reference an exercise library 166 containing information about exercises being performed by a patient, such as how much movement of a body part is needed to count as a repetition or to achieve a body part movement goal, both of which can be customized for individual patients.

The exercise metrics 172 can further comprise gait analysis metrics for exercise programs that have the patient walk towards, away from, or in front of the camera for the PTaaS backend to perform gait analysis of the patient. Gait analysis metrics can comprise information indicating step cadence, step length, step symmetry, body posture and/or alignment during walking, compensatory patterns (e.g., limping), and movement of joints (e.g., knees, ankles, hips) during walking.

The pre-checks module 168 can perform any of the pre-checks discussed above, including light range, patient position, camera distance, plane alignment, side orientation, occlusion, starting pose, and starting angle checks. The pre-checks module 168 can determine feedback to be provided to a patient prior to the patient starting activity based on information representing video of the patient prior to performing the activity. The information representing video of the user prior to performing the activity provided to the pre-checks module 168 can comprise 2D skeleton information provided by the person detection/2D skeleton extraction module 156 and body angle and body part movement information provided by the MSK exercise repetition count module 164. The pre-checks module 168 can send to the patient application 108 information representing feedback to be provided to the patient prior to the patient starting the activity (as feedback information 130). The information representing the feedback to be provided to the patient prior to the patient starting the activity can be any feedback relating to pre-checks discussed above (e.g., feedback indicating poor lighting conditions, the user is to move left or right, the user is to move forward or backward, the user is to position themselves in a starting pose, the user is to position a body part at a starting angle).

The live checks module 170 can perform any of the live checks discussed above, including correct limb movement, correct exercise, and form and posture checks. The live checks module 170 can determine feedback to be provided to a patient during performance of the activity based on information representing video of the patient performing the activity. The information representing video of the patient performing the activity provided to the live checks module 170 can comprise 2D skeleton information provided by the person detection/2D skeleton extraction module 156 and body angle and body part movement information provided by the MSK exercise repetition count module 164. The live checks module 170 can send to the patient application 108 information representing feedback to be provided to the patient during performance of the activity (as feedback information 130). The information representing feedback to be provided to the patient during the activity can be any feedback relating to live checks described above (e.g., feedback indicating an incorrect activity is being performed, an incorrect limb is being moved, an adjustment is to be made to the form or posture of the user, how much a patient is to adjust a range of motion of a body part).

The individual checks performed by the pre-checks module 168 (e.g., starting position check) and the live checks module 170 (e.g., correct exercise check) can be implemented with machine learning models, deep learning models, or other suitable models. As the PTaaS backend 116 can be implemented as a cloud-based service, the computational resources available to the PTaaS backend 116 can be scaled as needed. Thus, multiple checks can be performed by the pre-checks module 168 and the live checks module 170 for an exercise while still being able to provide real-time feedback to the patient. In some embodiments, the PTaaS backend 116 can perform multiple checks performed by the pre-checks module 168 and the live checks module 170 in parallel, by running multiple checks in parallel on the same computing device, or by running multiple checks in parallel on separate computing devices.

The feedback provided to the patient application by the live checks module 170 and the pre-checks module 168 is provided in real-time. In some embodiments, feedback associated with live checks can be provided as soon as the PTaaS backend detects the form or posture deficiency, after detection of completion of a repetition of an activity, or after detection of completion of a set of the activity.

In some embodiments, the PTaaS backend 116 is a cloud-based service that executes on one or more computing devices, such as one or more servers located in a data center. As such, a camera 104 can send real-time patient exercise video to one or more remote computing devices (e.g., remote servers) implementing the PTaaS backend 116 over one or more networks, such as the Internet. A patient application 108 receives information from the PTaaS backend over the one or more networks from the one or more remote computing systems hosting the PTaaS backend. Further, the PTaaS backend 116, being implemented as a scalable cloud-based service is not encumbered by the limited resources of an edge device (such as patient device 105) to perform patient exercise video analysis. The PTaaS backend 116 can utilize a greater amount of compute resources to implement a physical therapy assistant-as-a-service owing to its ability to utilize more powerful and/or a greater number of processors than is typically available at an edge computing device such as a tablet, personal laptop computer, or smartphone. This can allow the PTaaS backend 116 to perform more checks on real-time patient exercise video as well as more complex checks than could be performed on mobile edge computing devices.

In some embodiments, the checks implemented by the pre-checks module 168 and the live checks module 170 are expandable. That is, the pre-checks module 168 and the live checks module 170 can be updated to implement additional checks for existing or new exercises as the new checks become available. Further, the live checks and pre-checks performed for an exercise can be customized for individual patients. For example, in some embodiments, the pre-checks module 168 and the live checks module 170 can reference an exercise program store 167 that stores activity program profiles for individual patients, with an activity program profile capable of indicating which checks are to be performed for an exercise within an exercise program for a particular patient. Thus, as part of providing a PTaaS platform, a PTaaS backend can determine which pre-checks and live checks for an exercise are to be performed on a real-time patient exercise video based on an activity program profile associated with the user.

Patient-identifying information can be supplied from the patient application 108 to the PTaaS backend 116 during an exercise session. Which checks are to be performed for a particular exercise for a particular patient can be specified by a clinician via the clinician portal 112. Thus, in embodiments where the checks that a PTaaS backend performs on real-time patient exercise video is customizable, the PTaaS backend may perform fewer live checks than the live checks module 170 can perform and/or fewer pre-checks than the pre-checks module 168 is capable of performing. The pre-checks module 168 and the live checks module 170 may perform different checks for different patients.

The biomechanical insights module 148 can determine physical therapy insights based on exercise metrics 172 generated by the MSK exercise repetition count module 164. The biomechanical insights module 148 comprises an analytics aggregation module 174, a longitudinal patient metrics module 176, a physical therapy insights module 178, an exercise compliance tracking module 181, and a patient metrics store 182. The analytics aggregation module 174 aggregates exercise metrics 172 for a patient and can store them in the patient metrics store 182. The exercise compliance tracking module 181 can generate metrics and results that are provided to the patient application 108 (as metrics and results information 136) as well as to the clinician portal 112 indicating how well the patient is complying with an exercise program. The metrics and results information 136 can comprise information indicating, for example, a patient's range of motion for individual repetitions in a set, how many or what percentage of repetitions in a set or all sets for an exercise the patient performed the exercise without any live check errors (i.e., no form or posture deficiencies), how many repetitions, sets, and exercises a patient performed relative the repetition, goal, and exercise goals set out for the patient in the program, as well as addition exercise metrics and results relating to patient exercise performance.

The longitudinal patient metrics module 176 can generate patient longitudinal metrics based on a patient's performance of exercises over time, such as metrics indicating a patient's trend on how they are performing over time (e.g., repetition completion percentage trends, maximum range of motion trends). These longitudinal metrics can be stored in the patient metrics store 182. The physical therapy insights module 178 can extract insights from longitudinal metrics, such as whether there have been improvements in a patient's mobility, strength, endurance, and/or range of motion; whether the patient is adhering to a prescribed exercise program, and if there any days or times when the patient adherence is to an exercise program is higher or lower; whether the patient has reached a plateau (progress has stalled or slowed down); predictions as to when the patient may reach certain goals (such as regaining functionality); and how much longer the patient may need to continue physical therapy given their rate of progress toward program goals. These insights may also be stored in the patient metrics store 182.

Any of the metrics and insights stored in the patient metrics store 182 (including exercise metrics 172 and any of the metrics and insights generated by the biomechanical insights module 148) can be provided to the clinician portal 112 as metrics 184 and insights 188. In some of the embodiments, any of the metrics 184 and insights 188 can also be provided to the patient application 108 and displayed to the patient.

The clinician portal 112 can further allow a physical therapy clinician to provide feedback to a patient after assessing the patient's exercise performance and progress after reviewing the patient's exercise videos and any exercise metrics and insights provided by the PTaaS backend 116. Through the clinician portal 112, the clinician can provide verbal, visual, or textual feedback on the patient's progress or alter their exercise program (by adding or removing exercises, adding or removing checks to be performed for an exercise, etc.). This information can be provided to the patient via the patient application 108.

The clinician portal 112 allows a clinician to assemble an in-clinic/home exercise program for a patient and view the patient's performance of exercises along with metrics and insights regarding the patient's performance of the exercises. The clinician portal 112 can be an application running a computing device, such as a stand-alone application or a web-based application operating through a web browser running on a computing device. The clinician portal 112 can comprise a patient dashboard 186 within which a clinician can perform tasks for a specific patient (e.g., assemble an exercise program, view patient exercise videos, view reports and/or exercise metrics generated by a PTaaS backend) to assess a patient's progress. To establish an exercise program for a patient, a clinician can provide user input selecting one or more exercises to be performed as part of the exercise program. The clinician can further provide user input selecting one or more checks (pre-checks, live checks) associated with the exercise to be performed in real-time while the patient is preparing to perform or performing the exercise. The selection of one or more checks can comprise the clinician being presented with a set of checks associated with the exercise and the clinician selecting fewer than all the presented checks. The clinician can also select additional exercise program information, such as the number of sets, the number of repetitions to be performed in each set, a target body angle, a hold time for each exercise, a time limit for each set, and/or a rest time for between sets for each exercise. After selection of exercises and real-time checks to be performed for each exercise in the program, information indicating the exercise and the real-time checks to be performed for each exercise, along with the additional exercise program information can be sent to the PTaaS backend 116, where it is stored as an exercise program associated with a particular patient in the exercise program store 167.

A clinician can access the clinician portal 112 to view patient exercise videos with 2D skeleton overlays stored in the video content store 158. A patient exercise video can be viewed along with metrics associated with the patient exercise video, such as time-series metrics (e.g., knee angle) mapped to any video frame of post-exercise metrics. These metrics can be metrics 184 provided by the PTaaS backend 116.

FIG. 8 illustrates an example clinician portal display output. The display output 800 comprises a video 804 of a patient performing seated knee extensions. The video 804 comprises a 2D skeleton overlay over the patient's right leg and hip. A clinician can play, pause, advance, or rewind the video as desired. A graph 808 illustrates the patient's right knee angle over time during performance of the exercise by the patient. The graph 808 comprises a goal line 812 indicating a goal knee angle set for the exercise by the clinician.

The clinician can view any other exercise metrics or physical therapist insights that are generated by the PTaaS backend 116 during analysis of patient exercise videos, such as longitudinal metrics and physical therapy insights related to the patient's performance of an exercise over time, and anomaly detection. For example, anomalies detected by the PTaaS backend 116 during the presentation of an exercise can be indicated on a graph indicating a patient's performance of an exercise, such as by displaying information indicating the type of anomaly detected and when the anomaly was detected.

The PTaaS platform 100 can further comprise a PTaaS administrator portal (not shown). The PTaaS administrator portal can manage patient and clinician authorization to use the PTaaS platform, manage patient and clinician profiles, add pre-checks and live-checks that can be performed by the PTaaS backend for an exercise, add a new report that can be generated for a clinician, etc. In some embodiments, the PTaaS platform 100 can further perform patient and/or camera authorization and authentication. For example, the PTaaS backend 116 can receive patient-identifying information from the camera (or patient device containing the camera) or perform facial recognition on the patient's face in a patient exercise video and determine if the patient is authorized to use the PTaaS platform. The PTaaS backend 116 may similarly receive camera-identifying information from the camera (or patient device containing the camera) and determine whether the camera is authorized to send real-time patient exercise video to the PTaaS platform for analysis. If not, the PTaaS backend can send a message to the camera indicating that the camera is not approved for PTaaS platform use or simply ignore patient exercise video sent by the camera or patient device. The PTaaS backend 116 can further securely store patient information, such as personal identifying information (e.g., name, address), personal information (e.g., age, height, weight), and information identifying clinician-patient associations. By storing patient data securely in the cloud, the PTaaS platform can meet data security and patient privacy regulations. The PTaaS platform can allow healthcare institutes to control access to a patient's medical data. Further, the PTaaS platform can integrate with existing EMR (electronic medical record) systems to onboard clinicians and patients to an EMR system, retrieve a patient's exercise plan as documented in an EMR system, and provide patient exercise performance information as measured by the PTaaS platform in accordance with patient's exercise plan to an EMR system to be used by clinicians in an EMR setting to generate various reports that can be used by health institutes and insurance companies.

While the PTaaS technologies described herein allow for physical therapy services to be provided remotely, the PTaaS platform allows for patient-physical therapist interactions. A patient is still likely to visit a physical therapist for an initial assessment and can perform their exercise program in a clinical setting with a clinician providing live feedback (in addition to the real-time feedback that can supplied by the PTaaS platform during a clinic visit) and discuss their progress with a clinician during an in-person visit.

Although the technologies described herein are described as providing the automatic evaluation and feedback for patient performance of physical therapy exercises, they can be applied to non-physical therapy activities, such as ergonomics activities (e.g., stretches or arm movements made at a desk), wellness activities (e.g., yoga poses, Pilates activities), occupational therapy, or other activities, such as physical therapy patient assessments (e.g., assessments made during patient intake, such as gait analysis).

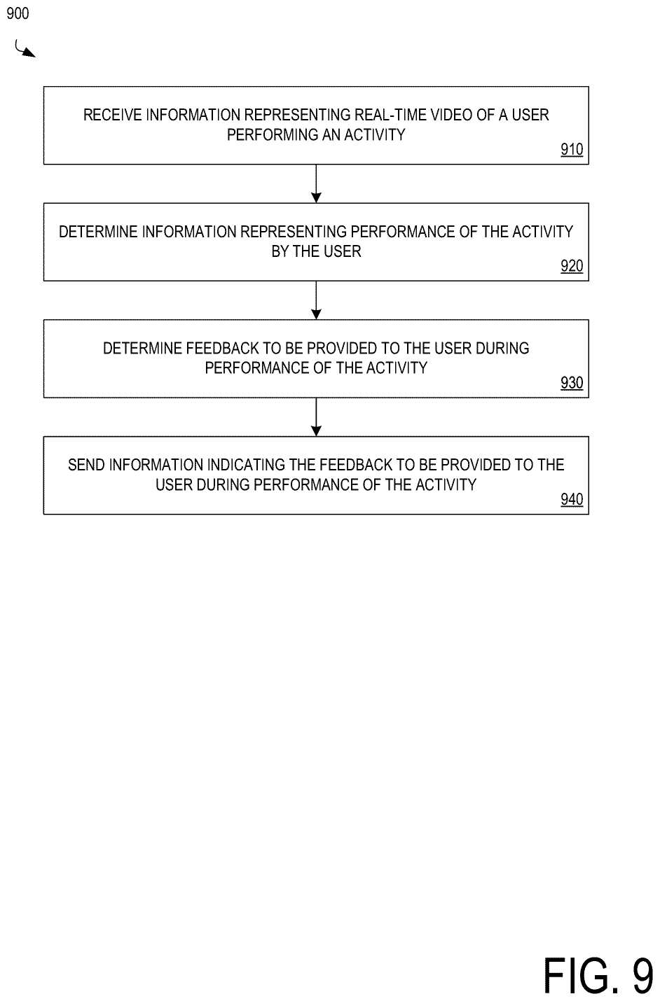

FIG. 9 is a first example method of a PTaaS platform providing real-time feedback to a patient performing physical therapy exercises. The method 900 can be performed by a PTaaS platform. At stage 910 in method 900, information is received at one or more first computing devices from a second computing device, representing real-time video of a user performing an activity, wherein the one or more first computing devices are remote to the second computing device. At stage 920, at the one or more first computing devices, information representing performance of the activity by the user based on the information representing real-time video of the user performing the activity is determined. At stage 930, at the one or more first computing devices, feedback to be provided to the user during performance of the activity based on the information representing real-time video of the user performing the activity is determined. At stage 940, information is sent from the second computing device to the one or more first computing devices, indicating the feedback to be provided to the user during performance of the activity.

In other embodiments, the method 900 can comprise one or more additional stages. For example, the method 900 can further comprise receiving, at the one or more first computing devices from the second computing device, information representing real-time video of the user prior to performing the activity; determining, at the one or more first computing devices, feedback to be provided to the user prior to the user starting the activity based on the information representing video of the user prior to performing the activity; and sending, from the one or more first computing devices computing device to the second computing devices, information representing the feedback to be provided to the user prior to the user starting the activity.

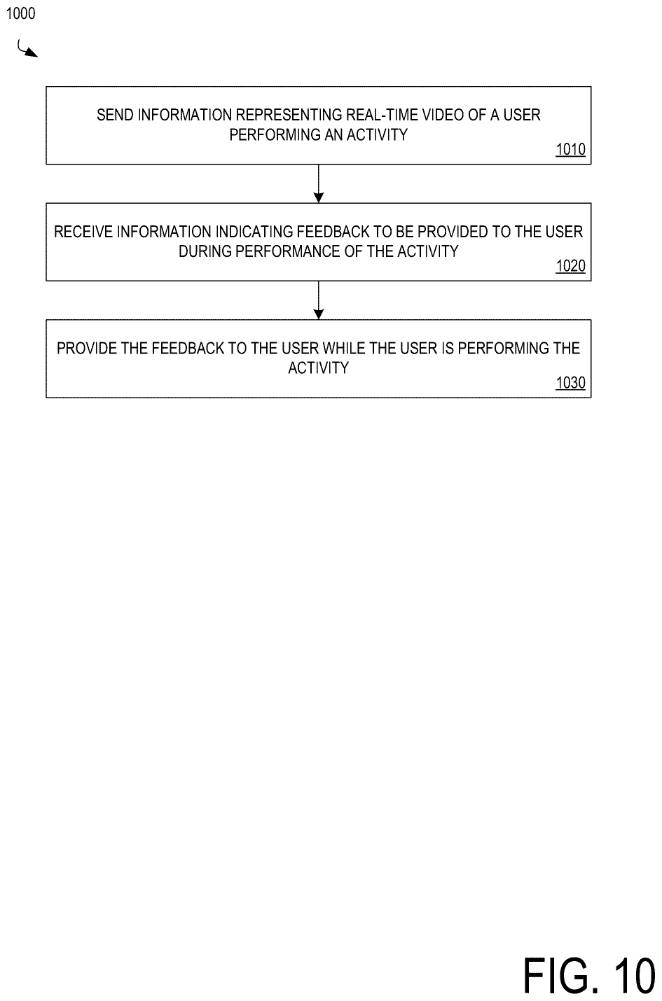

FIG. 10 is a first example method of providing feedback on real-time patient exercise video. The method 1000 can be performed by a patient computing device, such as a tablet. At stage 1010 in method 1000, information is sent to a first computing device from a second computing device or a third computing device (e.g., a stand-alone camera) representing real-time video of a user performing an activity, wherein the second computing device is remote to the first computing device. At stage 1020, at the second computing device, information indicating feedback to be provided to the user during performance of the activity is received. At stage 1030, feedback is provided at the second computing device to the user while the user is performing the activity.

In other embodiments, the method 1000 can comprise one or more additional stages. For example, the method 1000 can further comprise sending, to a first computing device from a second computing device, information representing real-time video of a user prior to performing the activity; receiving, at the second computing device, information indicating feedback to be provided to the user prior to performing the activity; and providing the feedback to the user prior to the user performing the activity.

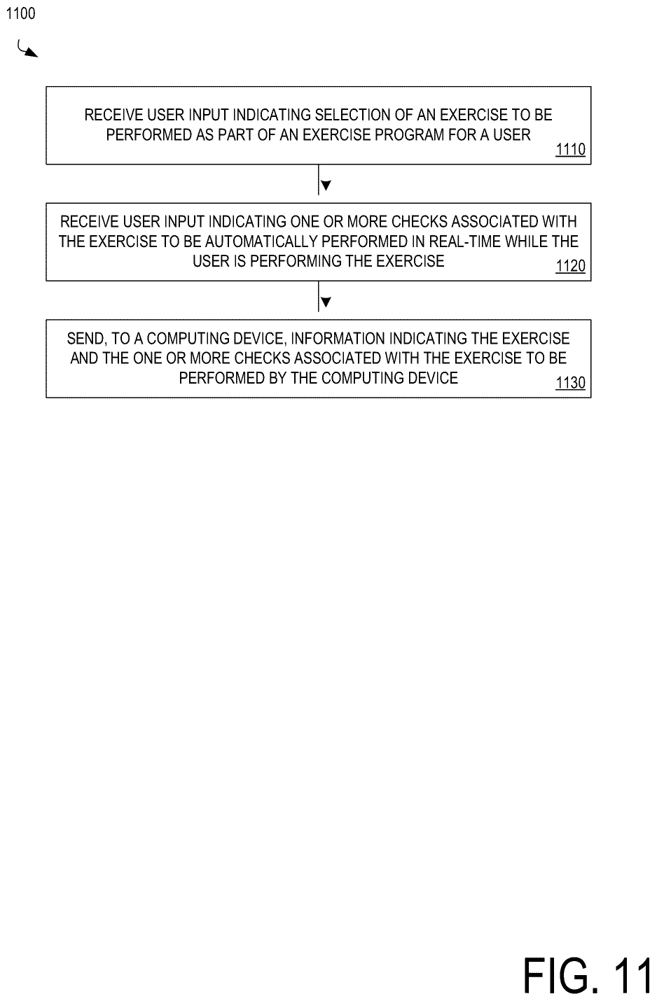

FIG. 11 is an example method of operating a clinician portal. At stage 1110 in method 1100, user input indicating selection of an exercise to be performed as part of an exercise program for a user is received at a first computing device. At stage 1120, user input indicating one or more checks associated with the exercise to be automatically performed in real-time while the user is performing the exercise is received at a first computing device. At stage 1130, information indicating the exercise and the one or more checks associated with the exercise to be performed by a second computing device that is remote to the first computing device is sent from the first computing device to the second computing device.

In other embodiments, the method 1100 can comprise one or more additional stages. For example, the method 1100 can further comprise receiving video of the user performing the exercise, wherein the video comprises a two-dimensional skeleton overlay; and displaying the video of the user performing the exercise on a display of the first computing device.

It is to be understood that FIG. 1 illustrates one example of a set of modules that can be included in a PTaaS platform. In other embodiments, the one or more computing devices that host a PTaaS platform can have more or fewer modules than those shown in FIG. 1. Further, separate modules can be combined into a single module, and a single module can be split into multiple modules. Moreover, any of the modules shown in FIG. 1 can be part of an operating system or a hypervisor of any computing device that is part of a PTaaS platform, one or more software applications independent of the operating system or hypervisor, or operate at another software layer.

As used herein, the term “module” refers to logic that may be implemented in a hardware component or device, software or firmware running on a processor unit, or a combination thereof, to perform one or more operations consistent with the present disclosure. Software and firmware may be embodied as instructions and/or data stored on non-transitory computer-readable storage media. As used herein, the term “circuitry” can comprise, singly or in any combination, non-programmable (hardwired) circuitry, programmable circuitry such as processor units, state machine circuitry, and/or firmware that stores instructions executable by programmable circuitry. Modules described herein may, collectively or individually, be embodied as circuitry that forms a part of a computing system. Thus, any of the modules can be implemented as circuitry, such as pre-checks circuitry and live checks circuitry. A computing system referred to as being programmed to perform a method can be programmed to perform the method via software, hardware, firmware, or combinations thereof.

Any portion of the PTaaS technologies described herein can be performed by or implemented in any of a variety of computing systems, including mobile computing systems (e.g., smartphones, handheld computers, tablet computers, laptop computers, portable gaming consoles, 2-in-1 convertible computers, portable all-in-one computers), non-mobile computing systems (e.g., desktop computers, servers, workstations, stationary gaming consoles, smart televisions, rack-level computing solutions (e.g., blade, tray, or sled computing systems)), and embedded computing systems (e.g., computing systems that are part of a vehicle, smart home appliance, consumer electronics product or equipment, manufacturing equipment).

As used herein, the term “computing system” includes computing devices and includes systems comprising multiple discrete physical components. In some embodiments, the computing systems are located in a data center, such as an enterprise data center (e.g., a data center owned and operated by a company and typically located on company premises), managed services data center (e.g., a data center managed by a third party on behalf of a company), a co-located data center (e.g., a data center in which data center infrastructure is provided by the data center host and a company provides and manages their own data center components (servers, etc.)), cloud data center (e.g., a data center operated by a cloud services provider that hosts companies' applications and data), or an edge data center (e.g., a data center typically having a smaller footprint than other data center types, located close to the geographic area that it serves).

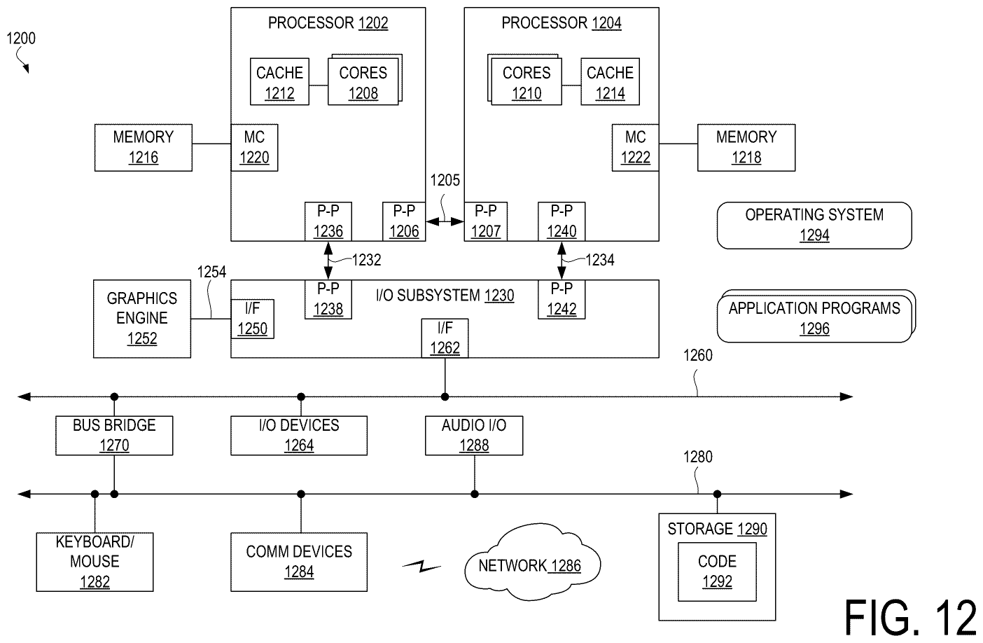

FIG. 12 is a block diagram of an example computing system in which technologies described herein may be implemented. Generally, components shown in FIG. 12 can communicate with other shown components, although not all connections are shown, for case of illustration. The computing system 1200 is a multiprocessor system comprising first processor unit 1202 and second processor unit 1204 comprising point-to-point (P-P) interconnects. A point-to-point (P-P) interface 1206 of the first processor unit 1202 is coupled to a point-to-point interface 1207 of the second processor unit 1204 via a point-to-point interconnection 1205. It is to be understood that any or all of the point-to-point interconnects illustrated in FIG. 12 can be alternatively implemented as a multi-drop bus, and that any or all buses illustrated in FIG. 12 could be replaced by point-to-point interconnects.

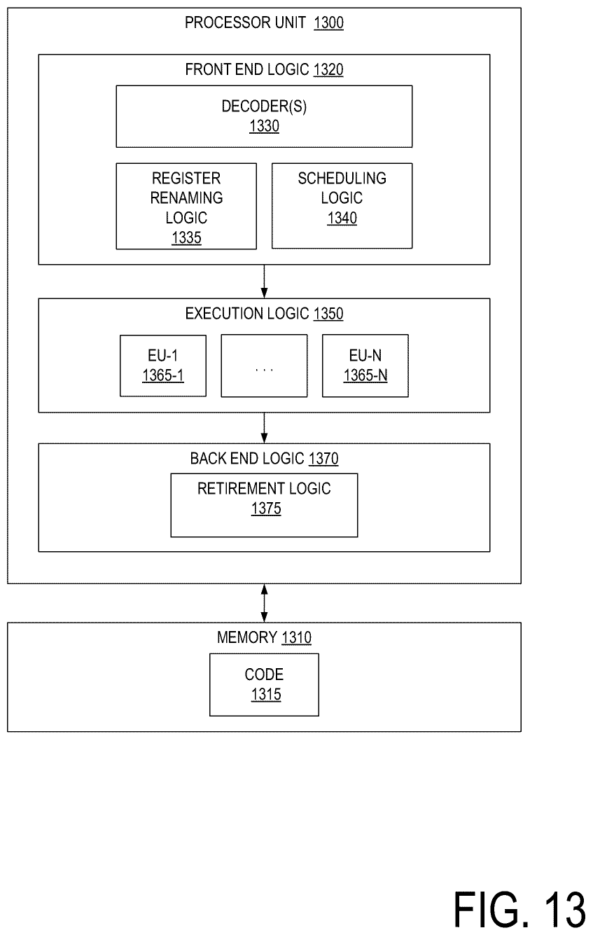

The first processor unit 1202 and second processor unit 1204 comprise multiple processor cores. The first processor unit 1202 comprises processor cores 1208 and the second processor unit 1204 comprises processor cores 1210. Processor cores 1208 and 1210 can execute computer-executable instructions in a manner similar to that discussed below in connection with FIG. 13, or other manners.

The first processor unit 1202 and the second processor unit 1204 further comprise cache memories 1212 and 1214, respectively. The cache memories 1212 and 1214 can store data (e.g., instructions) utilized by one or more components of the first processor unit 1202 and the second processor unit 1204, such as the processor cores 1208 and 1210. The cache memories 1212 and 1214 can be part of a memory hierarchy for the computing system 1200. For example, the cache memories 1212 can locally store data that is also stored in a first memory 1216 to allow for faster access to the data by the first processor unit 1202. In some embodiments, the cache memories 1212 and 1214 can comprise multiple cache memories that are a part of a memory hierarchy. The cache memories in the memory hierarchy can be at different cache memory levels, such as level 1 (L1), level 2 (L2), level 3 (L3), level 4 (L4), or other cache memory levels. In some embodiments, one or more levels of cache memory (e.g., L2, L3, L4) can be shared among multiple cores in a processor unit or among multiple processor units in an integrated circuit component. In some embodiments, the last level of cache memory in an integrated circuit component can be referred to as a last-level cache (LLC). One or more of the higher levels of cache levels (the smaller and faster cache memories) in the memory hierarchy can be located on the same integrated circuit die as a processor core and one or more of the lower cache levels (the larger and slower caches) can be located on one or more integrated circuit dies that are physically separate from the processor core integrated circuit dies.

Although the computing system 1200 is shown with two processor units, the computing system 1200 can comprise any number of processor units. Further, a processor unit can comprise any number of processor cores. A processor unit can take various forms such as a central processing unit (CPU), graphics processing unit (GPU), general-purpose GPU (GPGPU), accelerated processing unit (APU), field-programmable gate array (FPGA), neural network processing unit (NPU), data processor unit (DPU), accelerator (e.g., graphics accelerator, digital signal processor (DSP), compression accelerator, artificial intelligence (AI) accelerator), controller, or other type of processing unit. As such, the processor unit can be referred to as an XPU (or xPU). Further, a processor unit can comprise one or more of these various types of processing units. In some embodiments, the computing system comprises one processor unit with multiple cores, and in other embodiments, the computing system comprises a single processor unit with a single core. As used herein, the terms “processor unit” and “processing unit” can refer to any processor, processor core, component, module, engine, circuitry, or any other processing element described or referenced herein.

As used herein, the term “integrated circuit component” refers to a packaged or unpacked integrated circuit product. A packaged integrated circuit component comprises one or more integrated circuit dies mounted on a package substrate with the integrated circuit dies and package substrate encapsulated in a casing material, such as a metal, plastic, glass, or ceramic. In one example, a packaged integrated circuit component contains one or more processor units mounted on a substrate with an exterior surface of the substrate comprising a solder ball grid array (BGA). In one example of an unpackaged integrated circuit component, a single monolithic integrated circuit die comprises solder bumps attached to contacts on the die. The solder bumps allow the die to be directly attached to a printed circuit board. An integrated circuit component can comprise one or more of any computing system components described or referenced herein or any other computing system component, such as a processor unit (e.g., system-on-a-chip (SoC), processor core, graphics processor unit (GPU), accelerator, chipset processor), I/O controller, memory, or network interface controller.

In some embodiments, the computing system 1200 can comprise one or more processor units that are heterogeneous or asymmetric to another processor unit in the computing system. There can be a variety of differences between the processing units in a system in terms of a spectrum of metrics of merit including architectural, microarchitectural, thermal, power consumption characteristics, and the like. These differences can effectively manifest themselves as asymmetry and heterogeneity among the processor units in a system.

The first processor unit 1202 and the second processor unit 1204 can be located in a single integrated circuit component (such as a multi-chip package (MCP) or multi-chip module (MCM)) or they can be located in separate integrated circuit components. An integrated circuit component comprising one or more processor units can comprise additional components, such as embedded DRAM, stacked high bandwidth memory (HBM), shared cache memories (e.g., L3, L4, LLC), input/output (I/O) controllers, or memory controllers. Any of the additional components can be located on the same integrated circuit die as a processor unit, or on one or more integrated circuit dies separate from any integrated circuit die containing a processor unit. In some embodiments, these separate integrated circuit dies can be referred to as “chiplets”. In some embodiments, where there is heterogeneity or asymmetry among processor units in a computing system, the heterogeneity or asymmetric can be among processor units located in the same integrated circuit component. In embodiments where an integrated circuit component comprises multiple integrated circuit dies, interconnections between dies can be provided by a package substrate, one or more silicon interposers, one or more silicon bridges embedded in a package substrate (such as Intel® embedded multi-die interconnect bridges (EMIBs)), or combinations thereof.

The first processor unit 1202 further comprises first memory controller logic (first MC 1220) and the second processor unit 1204 further comprises second memory controller logic (second MC 1222). As shown in FIG. 12, a first memory 1216 coupled to the first processor unit 1202 is controlled by the first MC 1220 and a second memory 1218 coupled to the second processor unit 1204 is controlled by the second MC 1222. The first memory 1216 and the second memory 1218 can comprise various types of volatile memory (e.g., dynamic random-access memory (DRAM), static random-access memory (SRAM)) and/or non-volatile memory (e.g., flash memory, chalcogenide-based phase-change non-volatile memories). The first memory 1216 and the second memory 1218 can comprise one or more layers of a memory hierarchy of the computing system. While first MC 1220 and second MC 1222 are illustrated as being integrated into the first processor unit 1202 and the second processor unit 1204, in alternative embodiments, memory controller logic can be external to a processor unit.

The first processor unit 1202 and the second processor unit 1204 are coupled to an Input/Output subsystem 1230 (I/O subsystem) via point-to-point interconnections 1232 and 1234. The point-to-point interconnection 1232 connects a point-to-point interface 1236 of the first processor unit 1202 with a point-to-point interface 1238 of the Input/Output subsystem 1230, and the point-to-point interconnection 1234 connects a point-to-point interface 1240 of the second processor unit 1204 with a point-to-point interface 1242 of the Input/Output subsystem 1230. Input/Output subsystem 1230 further includes an interface 1250 to couple the Input/Output subsystem 1230 to a graphics engine 1252. The Input/Output subsystem 1230 and the graphics engine 1252 are coupled via a bus 1254.

The Input/Output subsystem 1230 is further coupled to a first bus 1260 via an interface 1262. The first bus 1260 can be a Peripheral Component Interconnect Express (PCIe) bus or any other type of bus. Various I/O devices 1264 can be coupled to the first bus 1260. A bus bridge 1270 can couple the first bus 1260 to a second bus 1280. In some embodiments, the second bus 1280 can be a low pin count (LPC) bus. Various devices can be coupled to the second bus 1280 including, for example, a keyboard/mouse 1282, audio I/O devices 1288, and a storage device 1290, such as a hard disk drive, solid-state drive, or another storage device for storing computer-executable instructions (or code 1292) or data. The code 1292 can comprise computer-executable instructions for performing methods described herein. Additional components that can be coupled to the second bus 1280 include one or more communication devices 1284, which can provide for communication between the computing system 1200 and one or more wired or wireless networks 1286 (e.g. Wi-Fi, cellular, or satellite networks) via one or more wired or wireless communication links (e.g., wire, cable, Ethernet connection, radio-frequency (RF) channel, infrared channel, Wi-Fi channel) using one or more communication standards (e.g., IEEE 502.11 standard and its supplements).

In embodiments where the one or more communication devices 1284 support wireless communication, the one or more communication devices 1284 can comprise wireless communication components coupled to one or more antennas to support communication between the computing system 1200 and external devices. The wireless communication components can support various wireless communication protocols and technologies such as Near Field Communication (NFC), IEEE 1002.11 (Wi-Fi) variants, WiMax, Bluetooth, Zigbee, 4G Long Term Evolution (LTE), Code Division Multiplexing Access (CDMA), Universal Mobile Telecommunication System (UMTS) and Global System for Mobile Telecommunication (GSM), and 5G broadband cellular technologies. In addition, the wireless modems can support communication with one or more cellular networks for data and voice communications within a single cellular network, between cellular networks, or between the computing system and a public switched telephone network (PSTN).

The computing system 1200 can comprise removable memory such as flash memory cards (e.g., SD (Secure Digital) cards), memory sticks, Subscriber Identity Module (SIM) cards). The memory in computing system 1200 (including cache memories 1212 and 1214, first memory 1216, second memory 1218, and storage device 1290) can store data and/or computer-executable instructions for executing an operating system 1294 and application programs 1296. Example data includes web pages, text messages, images, sound files, video data, patient data, and exercise metrics to be sent to and/or received from one or more network servers or other devices by the computing system 1200 via the one or more wired or wireless networks 1286, or for use by the computing system 1200. The computing system 1200 can also have access to external memory or storage (not shown) such as external hard drives or cloud-based storage.

The operating system 1294 can control the allocation and usage of the components illustrated in FIG. 12 and support the application programs 1296. The application programs 1296 can include common computing system applications (e.g., email applications, calendars, contact managers, web browsers, messaging applications) as well as other computing applications, such as patient application 108.

In some embodiments, a hypervisor (or virtual machine manager) operates on the operating system 1294 and the application programs 1296 operate within one or more virtual machines operating on the hypervisor. In these embodiments, the hypervisor is a type-2 or hosted hypervisor as it is running on the operating system 1294. In other hypervisor-based embodiments, the hypervisor is a type-1 or “bare-metal” hypervisor that runs directly on the platform resources of the computing system 1200 without an intervening operating system layer.

In some embodiments, the application programs 1296 can operate within one or more containers. A container is a running instance of a container image, which is a package of binary images for one or more of the application programs 1296 and any libraries, configuration settings, and any other information that the application programs 1296 need for execution. A container image can conform to any container image format, such as Docker®, Appc, or LXC container image formats. In container-based embodiments, a container runtime engine, such as Docker Engine, LXU, or an open container initiative (OCI)-compatible container runtime (e.g., Railcar, CRI-O) operates on the operating system (or virtual machine monitor) to provide an interface between the containers and the operating system 1294. An orchestrator can be responsible for management of the computing system 1200 and various container-related tasks such as deploying container images to the computing system 1200, monitoring the performance of deployed containers, and monitoring the utilization of the resources of the computing system 1200.

The computing system 1200 can support various additional input devices, such as a touchscreen, microphone, monoscopic camera, stereoscopic camera, trackball, touchpad, trackpad, proximity sensor, light sensor, electrocardiogram (ECG) sensor, PPG (photoplethysmogram) sensor, galvanic skin response sensor, and one or more output devices, such as one or more speakers or displays. Other possible input and output devices include piezoelectric and other haptic I/O devices. Any of the input or output devices can be internal to, external to, or removably attachable with the computing system 1200. External input and output devices can communicate with the computing system 1200 via wired or wireless connections.

In addition, the computing system 1200 can provide one or more natural user interfaces (NUIs). For example, the operating system 1294 or application programs 1296 can comprise speech recognition logic as part of a voice user interface that allows a user to operate the computing system 1200 via voice commands. Further, the computing system 1200 can comprise input devices and logic that allows a user to interact with computing the computing system 1200 via body, hand or face gestures. For example, the patient application 108 can prompt a user to wave their hand when they are ready to start an exercise.

The computing system 1200 can further include at least one input/output port comprising physical connectors (e.g., USB, FireWire, Ethernet, RS-232), a power supply (e.g., battery), a global satellite navigation system (GNSS) receiver (e.g., GPS receiver); a gyroscope; an accelerometer; and/or a compass. A GNSS receiver can be coupled to a GNSS antenna. The computing system 1200 can further comprise one or more additional antennas coupled to one or more additional receivers, transmitters, and/or transceivers to enable additional functions.

It is to be understood that FIG. 12 illustrates only one example computing system architecture. Computing systems based on alternative architectures can be used to implement technologies described herein. For example, instead of the first processor unit 1202, the second processor unit 1204, and the graphics engine 1252 being located on discrete integrated circuit dies, a computing system can comprise an SoC (system-on-a-chip) integrated circuit die on which multiple processors, a graphics engine, and additional components are incorporated. Further, a computing system can connect its constituent component via bus or point-to-point configurations different from that shown in FIG. 12. Moreover, the illustrated components in FIG. 12 are not required or all-inclusive, as shown components can be removed and other components added in alternative embodiments.

FIG. 13 is a block diagram of an example processor unit to execute computer-executable instructions as part of implementing technologies described herein. The processor unit 1300 can be a single-threaded core or a multithreaded core in that it may include more than one hardware thread context (or “logical processor”) per processor unit.

FIG. 13 also illustrates a memory 1310 coupled to the processor unit 1300. The memory 1310 can be any memory described herein or any other memory known to those of skill in the art. The memory 1310 can store computer-executable instructions 1315 (code) executable by the processor unit 1300.

The processor unit comprises front-end logic 1320 that receives instructions from the memory 1310. An instruction can be processed by one or more decoders 1330. The one or more decoders 1330 can generate as its output a micro-operation such as a fixed width micro-operation in a predefined format, or generate other instructions, microinstructions, or control signals, which reflect the original code instruction. The front-end logic 1320 further comprises register renaming logic 1335 and scheduling logic 1340, which generally allocate resources and queues operations corresponding to converting an instruction for execution.

The processor unit 1300 further comprises execution logic 1350, which comprises one or more execution units (EUs) (execution unit 1365-1 through execution unit 1365-N). Some processor unit embodiments can include a number of execution units dedicated to specific functions or sets of functions. Other embodiments can include only one execution unit or one execution unit that can perform a particular function. The execution logic 1350 performs the operations specified by code instructions. After completion of execution of the operations specified by the code instructions, back-end logic 1370 retires instructions using retirement logic 1375. In some embodiments, the processor unit 1300 allows out of order execution but requires in-order retirement of instructions. Retirement logic 1375 can take a variety of forms as known to those of skill in the art (e.g., re-order buffers or the like).

The processor unit 1300 is transformed during execution of instructions, at least in terms of the output generated by the one or more decoders 1330, hardware registers and tables utilized by the register renaming logic 1335, and any registers (not shown) modified by the execution logic 1350.

Any of the disclosed methods (or a portion thereof) can be implemented as computer-executable instructions or a computer program product. Such instructions can cause a computing system or one or more processor units capable of executing computer-executable instructions to perform any of the disclosed methods. As used herein, the term “computer” refers to any computing system, device, or machine described or mentioned herein as well as any other computing system, device, or machine capable of executing instructions. Thus, the term “computer-executable instruction” refers to instructions that can be executed by any computing system, device, or machine described or mentioned herein as well as any other computing system, device, or machine capable of executing instructions.

The computer-executable instructions or computer program products as well as any data created and/or used during implementation of the disclosed technologies can be stored on one or more tangible or non-transitory computer-readable storage media, such as volatile memory (e.g., DRAM, SRAM), non-volatile memory (e.g., flash memory, chalcogenide-based phase-change non-volatile memory) optical media discs (e.g., DVDs, CDs), and magnetic storage (e.g., magnetic tape storage, hard disk drives). Computer-readable storage media can be contained in computer-readable storage devices such as solid-state drives, USB flash drives, and memory modules. Alternatively, any of the methods disclosed herein (or a portion) thereof may be performed by hardware components comprising non-programmable circuitry. In some embodiments, any of the methods herein can be performed by a combination of non-programmable hardware components and one or more processing units executing computer-executable instructions stored on computer-readable storage media.

The computer-executable instructions can be part of, for example, an operating system of the computing system, an application stored locally to the computing system, or a remote application accessible to the computing system (e.g., via a web browser). Any of the methods described herein can be performed by computer-executable instructions performed by a single computing system or by one or more networked computing systems operating in a network environment. Computer-executable instructions and updates to the computer-executable instructions can be downloaded to a computing system from a remote server.

Further, it is to be understood that implementation of the disclosed technologies is not limited to any specific computer language or program. For instance, the disclosed technologies can be implemented by software written in C++, C#, Java, Perl, Python, JavaScript, Adobe Flash, C#, assembly language, or any other programming language. Likewise, the disclosed technologies are not limited to any particular computer system or type of hardware.

Furthermore, any of the software-based embodiments (comprising, for example, computer-executable instructions for causing a computer to perform any of the disclosed methods) can be uploaded, downloaded, or remotely accessed through a suitable communication means. Such suitable communication means include, for example, the Internet, the World Wide Web, an intranet, cable (including fiber optic cable), magnetic communications, electromagnetic communications (including RF, microwave, ultrasonic, and infrared communications), electronic communications, or other such communication means.

As used in this application and the claims, a list of items joined by the term “and/or” can mean any combination of the listed items. For example, the phrase “A, B and/or C” can mean A; B; C; A and B; A and C; B and C; or A, B and C. As used in this application and the claims, a list of items joined by the term “at least one of”' can mean any combination of the listed terms. For example, the phrase “at least one of A, B or C” can mean A; B; C; A and B; A and C; B and C; or A, B, and C. Moreover, as used in this application and the claims, a list of items joined by the term “one or more of” can mean any combination of the listed terms. For example, the phrase “one or more of A, B and C” can mean A; B; C; A and B; A and C; B and C; or A, B, and C.

As used in this application and the claims, the phrase “individual of” or “respective of” following by a list of items recited or stated as having a trait, feature, etc. means that all of the items in the list possess the stated or recited trait, feature, etc. For example, the phrase “individual of A, B, or C, comprise a sidewall” or “respective of A, B, or C, comprise a sidewall” means that A comprises a sidewall, B comprises sidewall, and C comprises a sidewall.

The disclosed methods, apparatuses, and systems are not to be construed as limiting in any way. Instead, the present disclosure is directed toward all novel and nonobvious features and aspects of the various disclosed embodiments, alone and in various combinations and subcombinations with one another. The disclosed methods, apparatuses, and systems are not limited to any specific aspect or feature or combination thereof, nor do the disclosed embodiments require that any one or more specific advantages be present or problems be solved.

Theories of operation, scientific principles, or other theoretical descriptions presented herein in reference to the apparatuses or methods of this disclosure have been provided for the purposes of better understanding and are not intended to be limiting in scope. The apparatuses and methods in the appended claims are not limited to those apparatuses and methods that function in the manner described by such theories of operation.

Although the operations of some of the disclosed methods are described in a particular, sequential order for convenient presentation, it is to be understood that this manner of description encompasses rearrangement, unless a particular ordering is required by specific language set forth herein. For example, operations described sequentially may in some cases be rearranged or performed concurrently. Moreover, for the sake of simplicity, the attached figures may not show the various ways in which the disclosed methods can be used in conjunction with other methods.

The following examples pertain to additional embodiments of technologies disclosed herein.

Example 1 is a method comprising: receiving, at one or more first computing devices from a second computing device or a third computing device, information representing real-time video of a user performing an activity, wherein the one or more first computing devices are remote to the second computing device; determining, at the one or more first computing devices, information representing performance of the activity by the user based on the information representing real-time video of the user performing the activity; determining, at the one or more first computing devices, feedback to be provided to the user during performance of the activity based on the information representing real-time video of the user performing the activity; and sending, from the one or more first computing devices to the second computing device, information indicating the feedback to be provided to the user during performance of the activity.

Example 2 comprises the method of Example 1, wherein the activity is a physical therapy exercise.

Example 3 comprises the method of Example 1, wherein determining feedback to be provided to the user during performance of the activity is performed after detection of completion of a repetition of the activity.

Example 4 comprises the method of Example 1, wherein determining feedback to be provided to the user during performance of the activity is performed after detection of completion of a set of the activity.

Example 5 comprises the method of any one of Examples 1-4, further comprising, prior to determining feedback to be provided to the user during performance of the activity, determining that the user is not performing a correct activity at a point within an activity program based on the information representing the real-time video of the user performing the activity, wherein the feedback to be provided to the user during performance of the activity indicates an incorrect activity is being performed.

Example 6 comprises the method of any one of Examples 1-4, further comprising, prior to determining feedback to be provided to the user during performance of the activity, determining that the user is moving an incorrect limb for the activity based on the information representing the real-time video of the user performing the activity, wherein the feedback to be provided to the user during performance of the activity indicates an incorrect limb is being moved.

Example 7 comprises the method of any one of Examples 1-4, further comprising, prior to determining feedback to be provided to the user during performance of the activity, determining that the user is using improper form for the activity based on the information representing the real-time video of the user performing the activity, wherein the feedback to be provided to the user during performance of the activity indicates an adjustment to be made to a form of the user.

Example 8 comprises the method of any one of Examples 1-4, further comprising, prior to determining feedback to be provided to the user during performance of the activity, determining that the user has an incorrect posture for the activity based on the information representing the real-time video of the user performing the activity, wherein the feedback to be provided to the user during performance of the activity indicates an adjustment to be made to a posture of the user.

Example 9 comprises the method of Example 7 or 8, wherein the feedback to be provided to the user during performance of the activity further comprises information indicating a quantitative adjustment to a range of a motion of a body part.

Example 10 comprises the method of any one of Examples 1-4, further comprising, prior to determining feedback to be provided to the user during performance of the activity, determining that multiple people are in a camera field of view based on the real-time video of a user performing an activity, wherein the feedback to be provided to the user during performance of the activity indicates multiple people are detected in the camera field of view.

Example 11 comprises the method of any one of Examples 1-10, wherein determining information representing performance of the activity by the user based on the information representing real-time video of the user performing an activity comprises continuously determining a body part angle of a user while the user is performing the activity based on the information representing real-time video of the user performing an activity, wherein the method further comprises continuously sending information indicating the body part angle of the user to the second computing device while the user is performing the activity.

Example 12 comprises the method of any one of Examples 1-10, wherein determining information representing performance of the activity by the user based on the information representing real-time video of the user performing an activity comprises continuously determining multiple body part angles of the user while the user is performing the activity based on the information representing real-time video of the user performing an activity, wherein the method further comprises continuously sending information indicating the multiple body part angles of the user to the second computing device while the user is performing the activity.

Example 13 comprises the method of any one of Examples 1-12, wherein determining information representing performance of the activity by the user based on the information representing real-time video of the user performing an activity comprises continuously determining information indicating a graphic element associated with a body part of the user to be overlaid on the user or displayed in a vicinity of the user in the real-time video of the user performing an activity based on the information representing real-time video of the user performing an activity, the method further comprising continuously sending information indicating a graphic element associated with a body part of the user to be overlaid on the user or displayed in a vicinity of the user in the real-time video of the user performing an activity while the user is performing the activity.

Example 14 comprises the method of any one of Examples 1-11, wherein determining information representing performance of the activity by the user based on the information representing real-time video of the user performing an activity comprises continuously determining information indicating a plurality of graphic elements associated with multiple body parts of the user to be overlaid on the user or displayed in a vicinity of the user in real-time video of the user performing an activity based on the information representing real-time video of the user performing an activity, the method further comprising continuously sending information indicating a plurality of graphic element associated with multiple body parts of the user to be overlaid on the user or displayed in a vicinity of the user in the real-time video of the user performing an activity while the user is performing the activity.

Example 15 comprises the method of any one of Examples 1-14, wherein the feedback to be provided to the user during performance of the activity indicates a number of completed repetitions.

Example 16 comprises the method of any one of Examples 1-14, wherein the feedback to be provided to the user during performance of the activity indicates a number of completed sets.

Example 17 comprises the method of any one of Examples 1-16, further comprising: receiving, at the one or more first computing devices from the second computing device, information representing real-time video of the user prior to performing the activity; determining, at the one or more first computing devices, feedback to be provided to the user prior to the user starting the activity based on the information representing video of the user prior to performing the activity; and sending, from the one or more first computing devices to the second computing device, information representing the feedback to be provided to the user prior to the user starting the activity.

Example 18 comprises the method of Example 17, further comprising, prior to determining feedback to be provided to the user prior to the user starting the activity, determining that a lighting brightness in the real-time video of the user performing the activity is too low or too high, wherein the feedback to be provided to the user prior to the user starting the activity indicates poor lighting conditions.

Example 19 comprises the method of Example 17, further comprising, prior to determining feedback to be provided to the user prior to the user starting the activity, determining that the user is not positioned property with a camera field of view based on the information representing video of the user prior to performing the activity, wherein the feedback to be provided to the user prior to the user starting the activity indicates the user is to move left or right.

Example 20 comprises the method of Example 17, further comprising, prior to determining feedback to be provided to the user prior to the user starting the activity, determining that the user is not located at a proper distance from a camera based on the information representing video of the user prior to performing the activity, wherein the feedback to be provided to the user prior to the user starting the activity indicates the user is to move forward or backward.

Example 21 comprises the method of Example 17, further comprising, prior to determining feedback to be provided to the user prior to the user starting the activity, determining that an incorrect anatomical plane of the user is aligned with a camera based on the information representing video of the user prior to performing the activity, wherein the feedback to be provided to the user prior to the user starting the activity indicates the user is to orient themselves so that a proper anatomical plane of the user is oriented to the camera.

Example 22 comprises the method of Example 17, further comprising, prior to determining feedback to be provided to the user prior to the user starting the activity, determining that the user is showing an incorrect body side to a camera based on the information representing video of the user prior to performing the activity, wherein the feedback to be provided to the user prior to the user starting the activity indicates the user is to orient themselves so a specific side of their body is facing the camera.

Example 23 comprises the method of Example 17, further comprising, prior to determining feedback to be provided to the user prior to the user starting the activity, determining that the user is in an incorrect starting pose based on the information representing video of the user prior to performing the activity, wherein the feedback to be provided to the user prior to the user starting the activity indicates the user is to position themselves in a starting pose.

Example 24 comprises the method of Example 17, further comprising, prior to determining feedback to be provided to the user prior to the user starting the activity, determining that a body part of the user is not at a starting angle based on the information representing video of the user prior to performing the activity, wherein the feedback to be provided to the user prior to the user starting the activity indicates the user is to position the body part at the starting angle.

Example 25 comprises the method of Example 17, further comprising, prior to determining feedback to be provided to the user prior to the user starting the activity based on the information representing video of the user prior to performing the activity, determining that all pre-checks for the activity have been passed, wherein the feedback to be provided to the user prior to the user starting the activity indicates the user is to start the activity.

Example 26 comprises the method of Example 17, further comprising, prior to determining feedback to be provided to the user prior to the user starting the activity, determining that the user is at least partially occluded based on the information representing video of the user prior to performing the activity, wherein the feedback to be provided to the user prior to the user starting the activity indicates the user is at least partially occluded.

Example 28 comprises the method of any one of Examples 1-27, further comprising determining which pre-checks checks for an activity are to be performed for the activity based on an activity program profile associated with the user.

Example 29 comprises the method of any one of Examples 1-27, further comprising determining which live checks for an activity are to be performed for the activity based on an activity program profile associated with the user.

Example 30 comprises the method of any one of Examples 1-29, wherein the one or more first computing devices are accessible to the second computing device over one or more networks.

Example 31 comprises the method of any one of Examples 1-29, wherein the one or more first computing devices are located in a data center.

Example 32 comprises the method of any one of Examples 1-31, wherein determining feedback to be provided to the user during performance of the activity comprises performing a plurality of checks on the information representing performance of the activity; receiving, at the one or more first computing devices, instructions to perform a new check on the information representing performance of the activity; and adding the new check to the plurality of checks.

Example 33 is a computing system comprising: one or more processing units; and one or more computer-readable storage media storing instructions that, when executed, cause the one or more processing units to perform the method of any one of Examples 1-32.

Example 34 is one or more computer-readable storage media storing instructions that, when executed, cause a computing system to perform the method of any one of Examples 1-32.

Example 35 is a method comprising: sending, to a first computing device from a second computing device, information representing real-time video of a user performing an activity, wherein the second computing device is remote to the first computing device; receiving, at the second computing device, information indicating feedback to be provided to the user during performance of the activity; and providing, at the second computing device, the feedback to the user while the user is performing the activity.

Example 36 comprises the method of Example 35, wherein the feedback to be provided to the user while the user is performing the activity comprises audio feedback.

Example 37 comprises the method of Example 35, wherein the feedback to be provided

to the user while the user is performing the activity comprises verbal feedback.

Example 38 comprises the method of Example 35, wherein the feedback to be provided to the user while the user is performing the activity comprises graphical feedback.

Example 39 comprises the method of Example 35, wherein the feedback to be provided to the user while the user is performing the activity comprises textual feedback.

Example 40 comprises the method of Example 35, wherein the feedback is provided to the user after completion of a repetition.

Example 41 comprises the method of Example 35, wherein the feedback is provided to the user after completion of a set.

Example 42 comprises the method of any one of Examples 35-41, wherein the feedback to be provided to the user while the user is performing the activity indicates an incorrect activity is being performed.

Example 43 comprises the method of any one of Examples 35-41, wherein the feedback indicates an incorrect limb is being moved.

Example 44 comprises the method of any one of Examples 35-41, wherein the feedback to be provided to the user while the user is performing the activity indicates an adjustment to be made to a form of the user.

Example 45 comprises the method of any one of Examples 35-41, wherein the feedback to be provided to the user while the user is performing the activity indicates an adjustment to be made to a posture of the user.

Example 46 comprises the method of Example 44 or 45, wherein the feedback to be provided to the user while the user is performing the activity further comprises information indicating a quantitative adjustment to a range of a motion of a body part.

Example 47 comprises the method of any one of Examples 35-41, wherein the feedback to be provided to the user while the user is performing the activity indicates detection of multiple people.

Example 48 comprises the method of any one of Examples 35-41, further comprising: continuously receiving, at the second computing device, information indicating a body part angle of the user; and causing the body part angle to be displayed on a display.

Example 49 comprises the method of any one of Examples 35-41, further comprising: continuously receiving, at the second computing device, information indicating multiple body part angles of the user; and causing the multiple body part angles to be displayed on a display.

Example 50 comprises the method of any one of Examples 35-41, further comprising: displaying the real-time video of the user performing an exercise on a display of the second computing device; continuously receiving, at the second computing device, information indicating a graphic element associated with a body part of the user; and causing the graphic element to be displayed on the display, the graphic element to overlay the user or to be displayed in a vicinity of the user in the real-time video of the user performing the activity.

Example 51 comprises the method of any one of Examples 35-41, further comprising: displaying the real-time video of the user performing an exercise on a display of the second computing device; continuously receiving, at the second computing device, information indicating multiple graphic elements associated with multiple body parts of the user; and causing the multiple graphic elements to be displayed on the display of the second computing device, the multiple graphic elements to overlay the user or to be displayed in a vicinity of the user in the real-time video of the user performing the activity.

Example 52 comprises the method of any one of Examples 35-41, further comprising: sending, to a first computing device from a second computing device, information representing real-time video of a user prior to performing the activity; receiving, at the second computing device, information indicating feedback to be provided to the user prior to performing the activity; and providing the feedback to the user prior to the user performing the activity.

Example 53 comprises the method of Example 51, wherein the feedback to be provided to the user prior to the user starting the activity indicates poor lighting conditions.

Example 54 comprises the method of Example 51, wherein the feedback to be provided to the user prior to the user starting the activity indicates the user is to move left or right.

Example 55 comprises the method of Example 51, wherein the feedback to be provided to the user prior to the user starting the activity indicates the user is to move forward or backward.

Example 56 comprises the method of Example 51, wherein the feedback to be provided to the user prior to the user starting the activity indicates the user is to orient themselves so that a proper anatomical plane of the user is oriented to a camera that is capturing the real-time video of the user performing the exercise.

Example 57 comprises the method of Example 51, wherein the feedback to be provided to the user prior to the user starting the activity indicates the user is to orient themselves so a specific side of their body is facing a camera that is capturing the real-time video of the user performing the exercise.

Example 58 comprises the method of Example 51, wherein the feedback to be provided to the user prior to the user starting the activity indicates the user is to position themselves in a starting pose.

Example 59 comprises the method of Example 51, wherein the feedback to be provided to the user prior to the user starting the activity indicates the user is to position a body part at a starting angle.

Example 60 comprises the method of Example 51, wherein the feedback to be provided to the user prior to the user starting the activity indicates the user is to start the activity.

Example 61 comprises the method of Example 51, wherein the feedback to be provided to the user prior to the user starting the activity indicates the user is at least partially occluded.

Example 62 comprises the method of Example 51, wherein the second computing device is a laptop computer, a tablet, or a smartphone.

Example 63 is a computing system comprising: a display; one or more processing units; and one or more computer-readable storage media storing instructions that, when executed, cause the one or more processing units to: perform the method of any one of Examples 35-62; and display the real-time video of the user performing the activity on the display.

Example 64 is one or more computer-readable storage media storing instructions that, when executed, cause a computing system to perform the method of any one of Examples 35-63.

Example 65 is a method comprising: receiving, at a first computing device, user input indicating selection of an exercise to be performed as part of an exercise program for a user; receiving, at the first computing device, user input indicating one or more checks associated with the exercise to be automatically performed in real-time while the user is performing the exercise; and sending, from the first computing device to a second computing device that is remote to the first computing device, information indicating the exercise and the one or more checks associated with the exercise to be performed by the second computing device.

Example 66 comprises the method of Example 65, further comprising, at the first computing device: receiving video of the user performing the exercise, wherein the video comprises a two-dimensional skeleton overlay; and displaying the video of the user performing the exercise on a display of the first computing device.

Example 67 comprises the method of Example 66, further comprising, at the first computing device: receiving metrics associated with the video of the user performing the exercise; and displaying metrics associated with the video of the user performing the exercise on the display of the first computing device.

Example 68 comprises the method of Example 67, wherein the metrics comprise a body part angle value that reflects an angle of a body part shown in the video, the body part angle value changing as the video of the user performing the exercise is played.

Example 69 comprises the method of Example 67, wherein the metrics comprise longitudinal metrics associated with performance of the exercise by the user.

Example 70 comprises the method of Example 66, further comprising receiving a physical therapy insight regarding with performance of the exercise by the user.

Example 71 is a computing system comprising: one or more processing units; one or more computer-readable storage media storing instructions that, when executed, cause the one or more processing units to: perform the method of any one of Examples 65-70.

Example 72 is one or more computer-readable storage media storing instructions that, when executed, cause a computing system to perform the method of any one of Examples 65-70.

1. A method comprising:
receiving, at one or more first computing devices from a second computing device or a third computing device, information representing real-time video of a user performing an activity, wherein the one or more first computing devices are remote to the second computing device;
determining, at the one or more first computing devices, information representing performance of the activity by the user based on the information representing real-time video of the user performing the activity;
determining, at the one or more first computing devices, feedback to be provided to the user during performance of the activity based on the information representing real-time video of the user performing the activity; and
sending, from the one or more first computing devices to the second computing device, information indicating the feedback to be provided to the user during performance of the activity.

2. The method of claim 1, further comprising, prior to determining feedback to be provided to the user during performance of the activity, determining that the user is not performing a correct activity based on the information representing the real-time video of the user performing the activity, wherein the feedback to be provided to the user during performance of the activity indicates an incorrect activity is being performed.

3. The method of claim 1, further comprising, prior to determining feedback to be provided to the user during performance of the activity, determining that the user is using improper form for the activity based on the information representing the real-time video of the user performing the activity, wherein the feedback to be provided to the user during performance of the activity indicates an adjustment to be made to a form of the user.

4. The method of claim 1, further comprising, prior to determining feedback to be provided to the user during performance of the activity, determining that the user has an incorrect posture for the activity based on the information representing the real-time video of the user performing the activity, wherein the feedback to be provided to the user during performance of the activity indicates an adjustment to be made to a posture of the user.

5. The method of claim 1, wherein determining information representing performance of the activity by the user based on the information representing real-time video of the user performing an activity comprises continuously determining a body part angle of a user while the user is performing the activity based on the information representing real-time video of the user performing an activity, wherein the method further comprises continuously sending information indicating the body part angle of the user to the second computing device while the user is performing the activity.

6. The method of claim 5, further comprising, prior to determining feedback to be provided to the user prior to the user starting the activity, determining that the user is showing an incorrect body side to a camera based on the information representing video of the user prior to performing the activity, wherein the feedback to be provided to the user prior to the user starting the activity indicates the user is to orient themselves so a specific side of their body is facing the camera.

7. The method of claim 5 further comprising, prior to determining feedback to be provided to the user prior to the user starting the activity, determining that the user is in an incorrect starting pose based on the information representing video of the user prior to performing the activity, wherein the feedback to be provided to the user prior to the user starting the activity indicates the user is to position themselves in a starting pose.

8. The method of claim 1, wherein determining feedback to be provided to the user during performance of the activity comprises performing a plurality of checks on the information representing performance of the activity;
receiving, at the one or more first computing devices, instructions to perform a new check on the information representing performance of the activity; and
adding the new check to the plurality of checks.

9. One or more computer-readable storage media storing instructions that, when executed, cause a computing system to:
receive, at one or more first computing devices from a second computing device or a third computing device, information representing real-time video of a user performing an activity, wherein the one or more first computing devices are remote to the second computing device, wherein the activity is a physical therapy exercise;
determine, at the one or more first computing devices, information representing performance of the activity by the user based on the information representing real-time video of the user performing the activity;
determine, at the one or more first computing devices, feedback to be provided to the user during performance of the activity based on the information representing real-time video of the user performing the activity; and
send, from the one or more first computing devices to the second computing device, information indicating the feedback to be provided to the user during performance of the activity.

10. The one or more computer-readable storage media of claim 9, wherein the one or more computer-readable storage media, when executed, is to further cause the computing system to: prior to determining feedback to be provided to the user during performance of the activity, determine that the user is moving an incorrect limb for the activity based on the information representing the real-time video of the user performing the activity, wherein the feedback to be provided to the user during performance of the activity indicates an incorrect limb is being moved.

11. The one or more computer-readable storage media of claim 9, wherein the one or more computer-readable storage media, when executed, is to further cause the computing system to, prior to determining feedback to be provided to the user during performance of the activity, determine that the user is using improper form for the activity based on the information representing the real-time video of the user performing the activity, wherein the feedback to be provided to the user during performance of the activity indicates an adjustment to be made to a form of the user.

12. The one or more computer-readable storage media of claim 9, wherein the feedback to be provided to the user during performance of the activity further comprises information indicating a quantitative adjustment to a range of a motion of a body part.

13. The one or more computer-readable storage media of claim 9, wherein determining information representing performance of the activity by the user based on the information representing real-time video of the user performing an activity comprises continuously determining information indicating a graphic element associated with a body part of the user to be overlaid on the user or displayed in a vicinity of the user in the real-time video of the user performing an activity based on the information representing real-time video of the user performing an activity, the one or more computer-readable storage media, when executed, is to further cause the computing system to continuously send information indicating a graphic element associated with a body part of the user to be overlaid on the user or displayed in a vicinity of the user in the real-time video of the user performing an activity while the user is performing the activity.

14. The one or more computer-readable storage media of claim 9, the one or more computer-readable storage media, when executed, is to further cause the computing system to:
receive, at the one or more first computing devices from the second computing device, information representing real-time video of the user prior to performing the activity;
determine, at the one or more first computing devices, feedback to be provided to the user prior to the user starting the activity based on the information representing video of the user prior to performing the activity; and
send, from the one or more first computing devices to the second computing device, information representing the feedback to be provided to the user prior to the user starting the activity.

15. The one or more computer-readable storage media of claim 14, the one or more computer-readable storage media, when executed, is to further cause the computing system to, prior to determining feedback to be provided to the user prior to the user starting the activity, determine that a body part of the user is not at a starting angle based on the information representing video of the user prior to performing the activity, wherein the feedback to be provided to the user prior to the user starting the activity indicates the user is to position the body part at the starting angle.

16. The one or more computer-readable storage media of claim 9, the one or more computer-readable storage media, when executed, is to further cause the computing system to determine which live checks for an activity are to be performed for the activity based on an activity program profile associated with the user.

17. A method comprising:
sending, to a first computing device from a second computing device or a third computing device, information representing real-time video of a user performing an activity, wherein the second computing device is remote to the first computing device;
receiving, at the second computing device, information indicating feedback to be provided to the user during performance of the activity; and
providing, at the second computing device, the feedback to the user while the user is performing the activity.

18. The method of claim 17, wherein the feedback to be provided to the user while the user is performing the activity indicates an adjustment to be made to a form of the user.

19. The method of claim 17, further comprising:
continuously receiving, at the second computing device, information indicating a body part angle of the user; and
causing the body part angle to be displayed on a display.

20. The method of claim 17, further comprising:
sending, to a first computing device from a second computing device, information representing real-time video of a user prior to performing the activity;
receiving, at the second computing device, information indicating feedback to be provided to the user prior to performing the activity; and
providing the feedback to the user prior to the user performing the activity, wherein the feedback to be provided to the user prior to the user starting the activity indicates the user is to position themselves in a starting pose or position a body part at a starting angle.
